SEQUENCE LISTING (89 sequences)

SEQ ID NO: 1 - DNA, 23 bp, Arabidopsis thaliana
tgaaggatcccttgttctacctg

SEQ ID NO: 2 - DNA, 32 bp, Arabidopsis thaliana
ttcgcggccgcattaactcgggtcggtgaaat

SEQ ID NO: 3 - DNA, 37 bp, Arabidopsis thaliana
cacgtcgactctaaaggttacttatggccaaagagag

SEQ ID NO: 4 - DNA, 32 bp, Arabidopsis thaliana
ttcgcggccgcagccactctccctctgacaat

SEQ ID NO: 5 - DNA, 32 bp, Arabidopsis thaliana
caccggtaccgttctttcacttgaactccctt

SEQ ID NO: 6 - DNA, 33 bp, Arabidopsis thaliana
gtcgactcgagcttcattaaacaaataagacga

SEQ ID NO: 7 - DNA, 35 bp, Arabidopsis thaliana
caccggatccatgggaagaaaaaaactagaaatca

SEQ ID NO: 8 - DNA, 36 bp, Arabidopsis thaliana
ttcgcggccgcgaattaagtagtgggagagtcaccg

SEQ ID NO: 9 - DNA, 33 bp, Arabidopsis thaliana
tgcaagcttcctaggaccggataagttcccttc

SEQ ID NO: 10 - DNA, 42 bp, Arabidopsis thaliana
tactagtctcgagctgttaatcagaaaaactcagattaatcg

SEQ ID NO: 11 - DNA, 31 bp, Arabidopsis thaliana
caccggtaccttggttaaaaatgccccacgc

SEQ ID NO: 12 - DNA, 30 bp, Arabidopsis thaliana
gtcgactcgagataacacaagaaagaagaa

SEQ ID NO: 13 - DNA, 32 bp, Arabidopsis thaliana
caccggtaccgtttgtgcactaactcaactct

SEQ ID NO: 14 - DNA, 33 bp, Arabidopsis thaliana
gtcgactcgagctgtaatgagagatagccatac

SEQ ID NO: 15 - DNA, 31 bp, Arabidopsis thaliana
caccggatccctgttcaatctctaattcgtc

SEQ ID NO: 16 - DNA, 37 bp, Arabidopsis thaliana
ttcgcggccgcgattactatttgtcttcagttaccca

SEQ ID NO: 17 - DNA, 30 bp, Arabidopsis thaliana
caccgtcgaccctttaatggccccattagt

SEQ ID NO: 18 - DNA, 39 bp, Arabidopsis thaliana
ttcgcggccgcattgatagttatgatcaaagacattgtt

SEQ ID NO: 19 - DNA, 30 bp, Arabidopsis thaliana
tggtctgtatcaagaagatgatcctatggc

SEQ ID NO: 20 - DNA, 23 bp, Arabidopsis thaliana
atggaaatgcttcatgcggcgaa

SEQ ID NO: 21 - DNA, 23 bp, Arabidopsis thaliana
agcagcctccatcacgagccaat

SEQ ID NO: 22 - DNA, 24 bp, Arabidopsis thaliana
caggttccttttgggcgttgaaat

SEQ ID NO: 23 - DNA, 23 bp, Arabidopsis thaliana
tggctcatggcgtcaggcttgtt

SEQ ID NO: 24 - DNA, 26 bp, Arabidopsis thaliana
ccaaatagagagacgaggatgagcgt

SEQ ID NO: 25 - DNA, 30 bp, Arabidopsis thaliana
agagatcacttcaacactcgtaaatttgcg

SEQ ID NO: 26 - DNA, 23 bp, Arabidopsis thaliana
tgcgtgcagcggtttctctttgt

SEQ ID NO: 27 - DNA, 25 bp, Arabidopsis thaliana
cgtattgtgttggtattgttccggc

SEQ ID NO: 28 - DNA, 30 bp, Arabidopsis thaliana
ggaagaccaagattgtagatctcagcaaac

SEQ ID NO: 29 - DNA, 25 bp, Arabidopsis thaliana
tggggatctcatgaaagcgaagttt

SEQ ID NO: 30 - DNA, 28 bp, Arabidopsis thaliana
cccaatgaacaattgcgtctctacttca

SEQ ID NO: 31 - DNA, 23 bp, Arabidopsis thaliana
gcagcgaagtacgggcatgagat

SEQ ID NO: 32 - DNA, 23 bp, Arabidopsis thaliana
cccacttaaatgctccacttggc

SEQ ID NO: 33 - DNA, 28 bp, Arabidopsis thaliana
atgcgcatgaagaagcattagctgatcc

SEQ ID NO: 34 - DNA, 25 bp, Arabidopsis thaliana
caagtaatcgcattctctccaccga

SEQ ID NO: 35 - DNA, 19 bp, Arabidopsis thaliana
cgccatccaagctgttctc

SEQ ID NO: 36 - DNA, 21 bp, Arabidopsis thaliana
caagacggaggatggcatgag

SEQ ID NO: 37 - DNA, 27 bp, Arabidopsis thaliana
tttctacaattgtcagagggagagtgg

SEQ ID NO: 38 - DNA, 25 bp, Arabidopsis thaliana
catcaccgttcgttactcgtatcat

SEQ ID NO: 39 - DNA, 24 bp, Arabidopsis thaliana
gacttagccgtgtgtctgaaaaga

SEQ ID NO: 40 - DNA, 22 bp, Arabidopsis thaliana
agcctctttaagcgtcgtcagt

SEQ ID NO: 41 - DNA, 24 bp, Arabidopsis thaliana
tgtgagtatcgatgctcttgttca

SEQ ID NO: 42 - DNA, 24 bp, Arabidopsis thaliana
ttcaacatgagttcggtcttcttg

SEQ ID NO: 43 - DNA, 25 bp, Arabidopsis thaliana
ggtgataaggatgccaaggaggttg

SEQ ID NO: 44 - DNA, 28 bp, Arabidopsis thaliana
gtccatactcgagttgtaatccacaagg

SEQ ID NO: 45 - DNA, 23 bp, Arabidopsis thaliana
aggagcaatggttggtgcgttcg

SEQ ID NO: 46 - DNA, 23 bp, Arabidopsis thaliana
caataagcgagttcctgacccca

SEQ ID NO: 47 - DNA, 21 bp, Arabidopsis thaliana
tggtggagaagayytyaggaa

SEQ ID NO: 48 - DNA, 26 bp, Arabidopsis thaliana
aatcccaarattcttatcctaaataa

SEQ ID NO: 49 - DNA, 20 bp, Arabidopsis thaliana
gtggctaccaagtgggagat

SEQ ID NO: 50 - DNA, 20 bp, Arabidopsis thaliana
taactcgggtcggtgaaatc

SEQ ID NO: 51 - DNA, 21 bp, Arabidopsis thaliana
ccaagagttgagattggtgga

SEQ ID NO: 52 - DNA, 21 bp, Arabidopsis thaliana
gggcatttttaaccaaggtct

SEQ ID NO: 53 - DNA, 28 bp, Arabidopsis thaliana
tcaagccagcctttaagatactctctgc

SEQ ID NO: 54 - DNA, 25 bp, Arabidopsis thaliana
gggttgctaggacttggaacatctg

SEQ ID NO: 55 - DNA, 20 bp, Arabidopsis thaliana
gctcaaacatgttgctcgaa

SEQ ID NO: 56 - DNA, 24 bp, Arabidopsis thaliana
tgcgatcagtaaaatacacagaca

SEQ ID NO: 57 - DNA, 21 bp, Arabidopsis thaliana
gatctacaatctcggccttcc

SEQ ID NO: 58 - DNA, 21 bp, Arabidopsis thaliana
atcatcaccgttcgttactcg

SEQ ID NO: 59 - DNA, 20 bp, Arabidopsis thaliana
tgggagggaaaaagatgtgt

SEQ ID NO: 60 - DNA, 22 bp, Arabidopsis thaliana
tggtaatggtgtttgtgaaacc

SEQ ID NO: 61 - DNA, 30 bp, Arabidopsis thaliana
gtgaaacaaattttcctgcagaatgtcact

SEQ ID NO: 62 - DNA, 33 bp, Arabidopsis thaliana
agtttttgaggcactaaaatctttgggtaaatc

SEQ ID NO: 63 - DNA, 33 bp, Arabidopsis thaliana
gattcttactgtaaagaacatggcattgagaga

SEQ ID NO: 64 - DNA, 29 bp, Arabidopsis thaliana
gatccaaatttcctgaggtgcttgtaacc

SEQ ID NO: 65 - DNA, 25 bp, Arabidopsis thaliana
cgtttcgctttccttagtgttagct

SEQ ID NO: 66 - DNA, 23 bp, Arabidopsis thaliana
agcgaacggatctagagactcac

SEQ ID NO: 67 - DNA, 23 bp, Arabidopsis thaliana
attgttactcaatgcatggctcc

SEQ ID NO: 68 - DNA, 21 bp, Arabidopsis thaliana
gccatcgtcttcagttccatt

SEQ ID NO: 69 - DNA, 618 bp, Arabidopsis thaliana
tgaaggatcccttgttctacctggtataactacaagaaaaggctgttttataaaatatatcatttcttctatgatttctatctcaactttttttttagtttttttcttttagaacgttttcgctttcgaattttttaagaattttttttttctttatatctctaaataatagttttaaaaacataacaagtgatactgtaatttttttttctctataaacttggcggtaccctacttttttcttatttttattaataatcttactttgttatgataaattatgttaatatatggcaaaactaaatatatatgaaactactgattactcattatcataattaatatctttgtataaaagtaaataatatttagtcgagagaggtatcttgttaaaggaaataaatcaaaaattagtggctaccaagtgggagatataatttggaatatttccagtgtattagtgtggtgggtttggaataccacaaacagaaataaaaagaaagaaaaatatgaaataagacgacaatgtgtgatgtacgtagaatcagttttagattctagtacatcaatagacaagaaaaagattgtggttatgatttcaccgacccgagttaatgc

SEQ ID NO: 70 - DNA, 815 bp, Arabidopsis thaliana
gttctttcacttgaactcccttttgtctcttttcttctttagtttcttcagtgcttcttacaccttcttttttaaaatagaaattattttccttttttggggtatactgaaaatatttcttgggcatgcagagaccttggttaaaaatgccccacgctttccttttctctgtttttttatgatttatttggttttactttatgatacccaaatcaaaaactaatttatatttcatttcttttttcatgaagatggacccataaaatatttcttcctatcctaaattaaatagagataaatttatgatctatcccaaattttttccaccaacttcttgcataagtgattattatttaggataagaatcttgggatttttctttgttcctcctacctaataatttaatttgcatttgtttttttttgtttttttttaagtataattttttactatactttgaaaatggcatttttgaaatatcctttttgctaattaaaatatctgcaaaaagatatagaaaattgatgttcattaaaacaaatatatatattgatgaatctctgttgtggaatatttgaaaactgtttgaataaggatattctgatattcaagccagcctttaagatactctctgctatatatagacatgtagctactaccttttttctattcatagttttcttcttctttcttgtgttatctcattttccaaacttcaaaaaagaaaaagaaaaaaagaccttttgctttcttgatttctttgaaaatgataatcttatcttcttataattcttcgtcttatttgtttaatgaag

SEQ ID NO: 71 - DNA, 570 bp, Arabidopsis thaliana
accttggttaaaaatgccccacgctttccttttctctgtttttttatgatttatttggttttactttatgatacccaaatcaaaaactaatttatatttcatttcttttttcatgaagatggacccataaaatatttcttcctatcctaaattaaatagagataaatttatgatctatcccaaattttttccaccaacttcttgcataagtgattattatttaggataagaatcttgggatttttctttgttcctcctacctaataatttaatttgcatttgtttttttttgtttttttttaagtataattttttactatactttgaaaatggcatttttgaaatatcctttttgctaattaaaatatctgcaaaaagatatagaaaattgatgttcattaaaacaaatatatatattgatgaatctctgttgtggaatatttgaaaactgtttgaataaggatattctgatattcaagccagcctttaagatactctctgctatatatagacatgtagctactaccttttttctattcatagttttcttcttctttcttgtgttatctc

SEQ ID NO: 72 - DNA, 713 bp, Arabidopsis thaliana
gtttgtgcactaactcaactctttaattaattcacttttaagttatagcatagctcaaacatgttgctcgaattatatatatagacactcaactacacatgtaaaactggacatgtattttggatactttgaaattgagtagatcacttataacttaagactcaaacattttacatttaatagaaggagacatataaaatcaacttcgagagtgcgatgcatcaatttgtctcccaaaaaagcccacacccaagctaaattgacatattttgttcaaaaactttactatgtgtaatgtaaatatgtattatgtatgtctgtgtattttactgatcgcacatagattctatagaatgtataggacgtgacttgaactaagatttgtttttttcactttaaagtggtctttgtacgggaatatcattgagcttaaaatagcctgatgacatcatcattgttccttgaagtgagcaggactaggtttcctgttatatcactttttatttttatttatatattcatgcacttgaatgttattgcatgttttgctagtcacttgcactagtaatctaggaattagttacgttgctattatttttttgtaagaaaataaagtttaagtagcatttcaattctgtcaaaaaaagaagtagcatttcaattatgttgtggtgccatagcttaaacatgtgtatggctatctctcattacag

SEQ ID NO: 73 - DNA, 434 bp, Arabidopsis thaliana
actctaaaggttacttatggccaaagagaggtgactaatggcttggatctaaggccttctcaggttcaaaacaagccaagagttgagattggtggagaagacctcaggaacttctatactttggttatggtggatccagatgttccaagtcctagcaaccctcacctccgagaatatctccattggttggtgactgatatccctgctacaactggaacaacctttggcaatgagattgtgtgttacgaaaatccaagtcccactgcaggaattcatcgtgtcgtgtttatattgtttcgacagcttggcaggcaaacagtgtatgcaccagggtggcgccagaacttcaacactcgcgagtttgctgagatctacaatctcggccttcccgtggccgcagttttctacaattgtcagagggagagtggctgcgg

SEQ ID NO: 74 - DNA, 384 bp, Arabidopsis thaliana
tccctgttcaatctctaattcgtcatgtcttattcgtttctttcattggaaaaaaaacactgaaaactagtttttgtttttccaactttcttggatcattaaataggattgctctctcttcaaacgtatttttgttggatctctcctcttcagttctagaagacttttttttttttccttttgttgagtgcatgatttcaaaaaatttcaattgaagaagtttgtgatattttgttgttttctttcttcaataagaggtgtgtccgcaattaagacaaaacaactcgctgtcatactcagttaaagtctttgttgctcgtgttcagattttcactagacgtcacaagtcaacttttgtgggtaactgaagacaaatagtaatcg

SEQ ID NO: 75 - DNA, 404 bp, Arabidopsis thaliana
gaccctttaatggccccattagtctgagtgttgattaaaagtataagctgactatactagatcttggaggaaatttgtatgtgcttcggccagaactctttgattataaacgtacaaaggataagtgactatactagatcttgccaaaaatatttcctatcatttagttatgactttcagtccgagacttgaatttacggagacaagggaacatataaatctaattctttgaatgacatagccaccatcatatgtgatttagcaattacatagtttgtaattctatatatcttttcacacgctaaataatgtgcattttcgcatttatgcatatataatggttcaaaatgtttttttggttttttgttctttctcaacaatgtctttgatcataactatcaatg

SEQ ID NO: 76 - DNA, 34 bp, Arabidopsis thaliana
tatctcgactccataatttctaacgaacatttgc

SEQ ID NO: 77 - DNA, 33 bp, Arabidopsis thaliana
tatgcggccgcagtcttcttcctccgcagccac

SEQ ID NO: 78 - DNA, 24 bp, Arabidopsis thaliana
ctgcatcagccgattatcatcacc

SEQ ID NO: 79 - DNA, 22 bp, Arabidopsis thaliana
catacctgttcaccgacgacgg

SEQ ID NO: 80 - DNA, 21 bp, Arabidopsis thaliana
tgatgatcttgtgctctgtcg

SEQ ID NO: 81 - DNA, 18 bp, Arabidopsis thaliana
atgacacccttggcttcg

SEQ ID NO: 82 - DNA, 25 bp, Arabidopsis thaliana
ttcctttattttccagtttggacag

SEQ ID NO: 83 - DNA, 20 bp, Arabidopsis thaliana
ttgcacgaccaggataattg

SEQ ID NO: 84 - DNA, 26 bp, Arabidopsis thaliana
gtttgtcgaccatataacacaagcgg

SEQ ID NO: 85 - DNA, 27 bp, Arabidopsis thaliana
cgaaagcgaaaacgttctaaaagaaaa

SEQ ID NO: 86 - DNA, 27 bp, Arabidopsis thaliana
aagattgtggttatgatttcaccgacc

SEQ ID NO: 87 - DNA, 27 bp, Arabidopsis thaliana
aacaaacaggtggtttctctgtgttga

SEQ ID NO: 88 - DNA, 21 bp, Arabidopsis thaliana
cacgtggcaatcttgtcttca

SEQ ID NO: 89 - DNA, 21 bp, Arabidopsis thaliana
agacgacgagaagagcgacgg

(19) United States
(12) Patent Application Publication
(10) Pub. No.: US 2016/0115494 A1
(43) Pub. Date: Apr. 28, 2016

(54) TRANSCRIPTIONAL GENE ACTIVATION

(71) Applicant: THE ROCKEFELLER UNIVERSITY, New York, NY (US)

(72) Inventors: Nam-Hai CHUA, New York, NY (US); Shulin DENG, New York, NY (US)

(73) Assignee: THE ROCKEFELLER UNIVERSITY, New York, NY (US)

(21) Appl. No.: 14/524,532

(22) Filed: Oct. 27, 2014

(51) Int. Cl. C12N 15/82; A01H 1/02

(52) U.S. Cl. CPC C12N 15/8216; C12N 15/8218; A01H 1/02

(57) ABSTRACT

The present invention relates to transcriptional gene activation. More specifically, the present invention relates to the methylation of the gene body using inverted repeat RNAs which results in the activation of gene expression without changing the tempo-spatial regulatory patterns of the gene expression.

### SEQUENCE SUBMISSION

The present application is being filed along with a Sequence Listing in electronic format. The Sequence Listing is entitled 2312134SequenceListing.txt, created on 13 Oct. 2014 and is 19 kb in size. The information in the electronic format of the Sequence Listing is incorporated herein by reference in their entirety.

### BACKGROUND OF THE INVENTION

The present invention relates to transcriptional gene activation. More specifically, the present invention relates to the methylation of the gene body using inverted repeat RNAs which results in the activation of gene expression without changing the tempo-spatial regulatory patterns of the gene expression.

The publications and other materials used herein to illuminate the background of the invention or provide additional details respecting the practice, are incorporated by reference, and for convenience are respectively grouped in the Bibliography.

Cytosine methylation is a heritable modification of DNA and plays an important role in regulating gene expression. Although it is known that DNA methylation in promoter regions correlates with transcriptional gene silencing (TGS), the role of gene body DNA methylation is less clear (Jones, 2012). In Arabidopsis, protein coding genes with intragenic DNA methylation are expressed at a moderate to high levels (Zhang et al., 2006; Zilberman et al., 2007), suggesting a positive role of gene body DNA methylation in transcription in plants. A positive correlation between gene body DNA methylation and gene expression levels has been recently observed in several genome-wide epigenomic studies in animals (Ball et al., 2009; Yu et al., 2013). However, in such genome wide association studies it is hard to distinguish the cause and effect relationship between DNA methylation status and transcript levels (Schtibeler, 2012; Jones et al., 2013).

In plants, DNA methylation at a specific locus can be introduced by expression of a homologous RNA through the RNA directed DNA methylation (RdDM) pathway (Wassenegger et al., 1994; Mette et al., 2000), making plants an ideal model system to study the function of DNA methylation. In the typical RdDM pathway, ˜24 nucleotide (nt) small RNAs (smRNAs) generated from inverted-repeat RNAs (IRs) by DCL3 are loaded onto AGO4, which together with plant specific RNA polymerase NRPE, recruits DNA methyltransferases (DRM1/2, MET1 and CMTS) to methylate DNA on the target locus (Matzke et al., 2009; Law and Jacobsen, 2010). Using RNAs targeting promoter regions, several Arabidopsis promoters can be methylated leading to TGS (Kinoshita et al., 2007; Deng et al., 2014). RNA targeting transcribed regions of genes can result in posttranscriptional gene silencing (PTGS) through smRNA/AGO1 mediated cleavage of mRNA. This event is usually associated with methylation of the targeted DNA regions, and in fact, gene body DNA methylation has been proposed to be an indicator of PTGS (Baulcombe, 1996). Several lines of experimental evidence indicate that transgene methylation is required for the maintenance of PTGS (Jones et al., 1999; Morel et al., 2000). In one case with Petunia hybrida it was reported that both silencing and activation of a target gene were observed when an RNA targeting the gene intronic region was used (Shibuya et al., 2009). However, the molecular mechanism remains unclear, because of the lack of genetic and/or biochemistry analysis. In a previous study on TGS of Arabidopsis endogens (Deng et al., 2014), some genes were activated when intronic regions were targeted

Flowering at an appropriate time is crucial for higher plants. In the long-day plant Arabidopsis, photoperiod signals are sensed by CONSTANS (CO) in leaves, where it promotes the expression of Flowering locus T (FT), the major component of florigen. The FT protein is transported to the shoot apical meristem, where it associates with the transcription factor FD to directly activate the floral identity gene AP1 (Turck et al., 2008). FT is also directly repressed by the MADS-box transcription factor, FLC (Helliwell et al., 2006).

A gene product can be expressed in a plant using CaMV 35S promoter or other constitutive promoters, such as ubiquitin, actin, and the like. These promoters have the disadvantage that they lack tissue specificity and quite often result in an undesirable phenotype or even lethality of plants. An alternative is to use tissue specific promoters, but in this case, the expression level is limited by the intrinsic promoter activity. It is desired to develop constructs and methods for transcriptional gene activation in plants without compromising tissue specificity of temporal expression pattern.

### SUMMARY OF THE INVENTION

The present invention relates to transcriptional gene activation. More specifically, the present invention relates to the methylation of the gene body using inverted repeat RNAs which results in the activation of gene expression without changing the tempo-spatial regulatory patterns of the gene expression.

As described and shown herein, using the FT locus as a model gene, evidence is provided that gene body DNA methylation facilitated transcription by blocking binding of a negative transcription factor and/or altering chromatin modification in Arabidopsis. More importantly, the tempo-spatial expression patterns of FT were maintained in the activated lines. A similar result was also obtained for other Arabidopsis genes.

The present invention provides methods and compositions for activating gene expression in plants while maintaining tissue specificity and temporal expression pattern. In certain embodiments, the activation of gene expression is provided by expression of a nucleic acid construct that produces a double stranded RNA molecule (dsRNA). In some embodiments the gene activation is provided by expression of a dsRNA fusion construct. In some embodiments of the invention, the dsRNA interferes with expression of a target gene described herein. In one embodiment RNA-mediated gene activation can be conferred by the expression of an inverted-repeat transgene cassette that generates a population of small interfering RNAs (siRNAs) (also sometimes referred to herein as small RNAs (smRNA)) derived from the dsRNA region of a transgene transcript.

Thus in one aspect, the present invention provides a nucleic acid construct which comprises one or more nucleic acid fragments. In one embodiment, the nucleic acid construct comprises one nucleic acid fragment. In another embodiment, the nucleic acid construct comprises two nucleic acid fragments. In a further embodiment, the nucleic acid construct comprises three nucleic acid fragments. In some embodiments, two or more nucleic acid fragments are linked together in the nucleic acid construct. In other embodiments, each nucleic acid fragment comprises sequences substantially homologous to target regions within introns of one, two or more target genes for activating the one, two or more target genes.

In some embodiments, the one or more nucleic acid fragments are present in the nucleic acid construct such that a dsRNA is produced, preferably the one or more nucleic acid fragments constitute a part of the dsRNA. In other embodiments, the one or more nucleic acid fragments are present in the nucleic acid construct such that a hairpin construct is produced upon expression of the one or more nucleic acid fragments and the one or more nucleic acid fragments constitute the stem of the hairpin construct or a part thereof. In one embodiment, the nucleic acid construct comprises one or more nucleic acid fragments in a sense orientation with respect to the promoter, a spacer sequence that can form a loop and the one or more nucleic acid fragments in an antisense orientation with respect to the promoter and preferably in reverse order with respect to the order of the one or more nucleic acid fragments in sense orientation. In another embodiment, the nucleic acid construct comprises one or more nucleic acid fragments in an antisense orientation with respect to the promoter, a spacer sequence that can form a loop and the one or more nucleic acid fragments in a sense orientation with respect to the promoter and preferably in reverse order with respect to the order of the one or more nucleic acid fragments in antisense orientation. In an additional embodiment, the nucleic acid construct comprises one or more nucleic acid fragments in a sense orientation with respect to the promoter, an intron and the one or more nucleic acid fragments in an antisense orientation with respect to the promoter and preferably in reverse order with respect to the order of the one or more nucleic acid fragments in sense orientation. In a further embodiment, the nucleic acid construct comprises one or more nucleic acid fragments in an antisense orientation with respect to the promoter, an intron and the one or more nucleic acid fragments in a sense orientation with respect to the promoter and preferably in reverse order with respect to the order of the one or more nucleic acid fragments in antisense orientation. In an embodiment where the nucleic acid construct comprises more than one nucleic acid fragments in sense and/or in antisense orientation, the nucleic acid fragments can be connected by means of a linker sequence which comprises at least four nucleotides. Furthermore, the linker sequence can be a spacer sequence that can form a loop or an intron. In some embodiments, the intron is removed from the nucleic acid construct during transcription to produce a loop-less hairpin (Smith et al., 2000).

In some embodiments, the nucleic acid construct further comprises a plant operable promoter operably linked to the one or more nucleic acid fragments. In one embodiment, a plant operable promoter is linked to each nucleic acid fragment. In another embodiment, a plant operable promoter is linked to two or more nucleic acid fragments that area linked together. In some embodiments, the plant operable promoter is heterologous to the one or more nucleic acid fragments. In some embodiments, the nucleic acid construct further comprises a plant operable terminator. In one embodiment, a plant operable terminator is linked to each nucleic acid fragment. In another embodiment, a plant operable terminator is linked to two or more nucleic acid fragments that area linked together. In some embodiments, the plant operable terminator is heterologous to the one or more nucleic acid fragments.

In other embodiments, the nucleic acid construct further comprises a selectable marker. In some embodiments, the selectable marker is part of a recombination marker free system. In one embodiment, the recombination marker free system is a Cre-lox recombination marker free system, a Zinc finger marker free system, a TALE nucleases marker free system or a CRISPR-Cas marker free system. In some embodiments, the recombination marker free system is positioned between the plant operable promoter and the one or more nucleic acid fragments.

In a second aspect, the present invention provides a transgenic plant comprising one or more nucleic acid constructs described herein in which of the target genes are activated in the transgenic plant. In some embodiments, the present invention provides any generation of plants having activated gene expression, including those described herein. In other embodiments, the present invention provides transgenic plant seed of any generation of such plants. In some embodiments, the present invention provides a method to prepare transgenic plants and seeds described herein.

In a third aspect, the present invention provides a method for activating gene expression in a plant described herein, the method comprising expressing in the plant at least one nucleic acid construct that expresses dsRNA targeting introns of target genes. In some embodiments, one, two or three nucleic acid constructs that express dsRNA targeting introns of target genes are used to confer activation of gene expression.

### BRIEF DESCRIPTION OF THE FIGURES

FIGS. 1a-1g show that an RNAi construct targeting FT intron 1 regions activates FT expression. FIG. 1a: Schematic diagrams showing the gene structure of FT. Dash lines, open boxes, solid boxes, and solid lines indicate promoter regions, 5′ untranslated regions (UTRs), coding regions, and intronic regions, respectively. Grey bars under the schematic gene structure show the targeted regions of each RNAi construct. FIG. 1b: Flowering phenotype of T1 RNAi transformants targeting the promoter (FTₚᵣₒ-Ri) and exon (FTₑₓ-Ri) of FT, EGFP RNAi plants were used as a control (EGFP-Ri). Each plant represented an independent line. Black bar=5 cm. FIG. 1c: Relative FT expression levels of controls (Col-0 and EGFP-Ri) and two lines each of RNAi construct targeting FT promoter (FTₚᵣₒ-Ri) or exon regions (FTₑₓ-Ri). FIG. 1d: Flowering phenotype of T1 RNAi transgenic plants targeting the FT first intron EGFP RNAi plants were used as a control (EGFP-Ri). Each plant represented an independent line. Bar=5 cm. FIG. 11e: Flowering times of FTᵢₙ₁-Ri lines. The number of rosette leaves at the time of flowering was measured. Data were presented as mean of 20 individuals±SD (standard deviation). FIG. 1f: FT mRNA levels in controls (Col-0 and EGFP-Ri) and three independent RNAi lines targeting FT intron 1 regions (FTᵢₙ₁-Ri). FIG. 1g: Real-time RT-PCR measured mRNA levels of flowering regulatory genes in FTᵢₙ₁-Ri lines. All quantitative RT-PCR data shown are means of three biological replicates±SD (FIGS. 1c, if and 1g).

FIGS. 2a-2f show that FTᵢₙ₁-Ri promotes FT expression through the RdDM pathway. FIG. 2a: Northern blot of small RNAs derived from the FT intron 1 region in FTᵢₙ₁-Ri plants (upper panel). MicroRNA 159 was used as a loading control (lower panel). Four independent FTᵢₙ₁-Ri transgenic lines together with two control plants were analyzed. FIG. 2b: Phenotype of ago]-27 and ago]-27;FTᵢₙ₁-Ri-6. Bar=5 cm. FIG. 2c: FT mRNA levels agol-27 and ago1-27;FTᵢₙ₁-Ri-6. FIG. 2d: Phenotype of AGO4; FTᵢₙ₁-Ri-6 and ago4-2;FTᵢₙ₁-Ri-6. Bar=5 cm. FIG. 2e: FT mRNA levels in AGO4;FTᵢₙ₁-Ri-6 and ago4-2;FTᵢₙ₁-Ri-6. FIG. 2f: DNA methylation at the FT locus by sequencing bisulfite-treated DNA. Upper panel shows the detection regions and lower panel shows the methylation levels of three independent FTᵢₙ₁-Ri transgenic lines and an EGFP RNAi control line. Bisulfite treatments were conducted twice using different DNA samples. Histograms show average values±SEM.

FIGS. 3a-3h show that methylation of FT CArG boxes blocks FLC binding but is not necessary for the transcriptional activation by the intronic RNAi of FT. FIG. 3a: Schematic diagrams showing the gene structure of FT and SOCl and predicted CArG boxes (*). Boxes indicate exon regions and lines indicate intron or intergenic regions. Grey bars beneath schematic gene structure diagrams indicate the detected regions in (FIG. 3b). FIG. 3b: FLC accumulation in FT and SOCl loci in Col-0 WT or FTᵢₙ₁-Ri-6 backgrounds. Target DNA from FLC-HA immunoprecipitates against input DNA were quantified by real time PCR. Data were further normalized to the enrichment level in WT. FIG. 3c: Schematic diagrams showing targeted regions of _{FTin1s-Ri} and FTᵢₙ₂-Ri (red bars). FIG. 3d: Phenotype of of T1 RNAi transgenic lines targeting partial FT intron 1 (FTᵢₙ₁ₛ-Ri) and intron 2 (FTᵢₙ₂-Ri). EGFP RNAi plants were used as controls (EGFP-Ri). Each plant represented an independent line. Blavk bar=5 cm. FIG. 3e: Flowering times of FT_{in1s-Ri} and FTᵢₙ₂-Ri lines. The number of rosette leaves at the time of flowering was measured. Data were presented as mean of 20 individuals±SD. FIG. 3f: FT mRNA levels of indicated lines. FIG. 3g: DNA methylation levels in FTᵢₙ₁ₛ-Ri-2. The methylation levels of two cytosines locate in the CArG boxes (grey box) and their nearest three cytosines upstream and downstream are shown. The numbers on the X-axis refer to the nucleotide positions 5′ or 3′ away from CArG boxes. FIG. 3h: FLC accumulation on FT and SOCl loci in WT or FTᵢₙ₁-Ri-6. Target DNA from FLC-HA immunoprecipitates against input DNA were quantified by real time PCR. Data were further normalized to the enrichment level in WT. All quantitative PCR data shown are means of three biological replicates±SD (FIGS. 3b, 3f and 3h).

FIGS. 4a-4d show histone modifications on the FT locus. FIG. 4a: Schematic diagram showing the gene structure of FT and primer pairs (P1 to P3) used for ChIP PCR. Three different activated lines (FTᵢₙ₁-Ri-1, FTᵢₙ₁-Ri-6, and FTᵢₙ₁ₛ-Ri-2), WT, and one EGFP RNAi control line (EGFP-Ri) were analyzed using anti-H3K4me3 (FIG. 4b), H3K9me3 (FIGS. 4c), and H3K9/14ac (FIG. 4d) antibody. For quantifications, relative enrichments against input were calculated, and relative enrichments are shown to those of the control, ATC7 (FIGS. 4b and 4c or Tai (FIG. 4b). All quantitative PCR data shown are means of three biological replicates±SD (FIGS. 4b and 4).

FIGS. 5a-5f show the maintenance of target gene tempo-spatial expression patters in plants activated by intronic RNAi. FIG. 5a: FT mRNA dynamics in the indicated seedlings over a 24 hr cycle. Relative expression levels were normalized to ZT (Zeitgeber time) Oh (at the beginning of a day). FIG. 5b: Relative expression levels of GUS mRNA detected by RT-qPCR. FIG. 5c: GUS staining of 2-week old gFT-GUS;XVE:FTᵢₙ₁-Ri seedlings grown on half strength MS medium with or without inducer. FIG. 5d: Activation of ATPT2/Pht];4 by a RNAi construct target on ATPT2/Pht1;4 intronic regions. Upper panel, a schematic diagram showing the ATPT2/Pht1;4 gene structure. Solid boxes indicate exon regions and black bars indicate intronic or intergenic regions. Grey bars under schematic diagrams indicate the target regions of RNAi. Lower panel, ATPT2 and ATPT1 expression levels in two EGFP-Ri lines and three ATPT2ᵢₙ-Ri lines detected by RT-qPCR. FIG. 5e: Activation of BMY1/RAM1 by a RNAi construct targeting BMYHRAM1 intronic regions. Upper panel, a schematic diagram showing the BMY1/RAM1 gene structure. Solid boxes indicate exon regions and black bars indicate intronic or intergenic regions. Bars under schematic diagrams indicate the target regions of RNAi. Lower panel, BMY1 and BMY2 expression levels in two EGFP-Ri lines and three BMY1 ᵢₙ-Ri lines detected by RT-qPCR. FIG. 5f: Tissue expression pattern of BMY1 detected by RT-qPCR. Data of RT-qPCR shown are means of three biological replicates±SD.

FIG. 6 shows expression levels of MFT, TSF, and TFL] in indicated lines examined by real time RT PCR. Data shown are means±SD.

FIGS. 7a and 7b show FT mRNA levels in T2 plants harboring UBQ10ₚᵣₒ:FTᵢₙ₁-Ri of WT (FIG. 7a), or ddc mutant (FIG. 7b) background. Nine lines and two UBQ10ₚᵣₒ:EGFP-Ri lines were investigated. Data shown are means±SD.

FIGS. 8a-8c show DNA methylation patterns on the FT first intronic regions in FTᵢₙ₁-Ri-1 (FIG. 8a), FTᵢₙ₁-Ri-3 (FIG. 8b), and FTᵢₙ₁-Ri-6 (FIG. 8c) plants. The numbers on the X-axis refer to the nucleotide positions downstream of the FT intron 1 first 5′ nt.

FIGS. 9a-9d show FLC accumulation in WT, FTᵢₙ₁-Ri-6, and FTᵢₙ₁ₛ-Ri-2 plants. FIG. 9a: Flowering phenotype of indicated plants. Bar=5 cm. FIG. 9b: Western blot to detect FLC-HA expression in WT, FTᵢₙ₁-Ri-6, and FTᵢₙ₁ₛ-Ri-2 plants (upper panel). Tubulin was used as loading control (lower panel). FIG. 9c and FIG. 9d were repeat experiments of FIGS. 3b and 3h, respectively, using different transgenic lines.

FIGS. 10a-10c show DNA methylation on the FT intronic region of FTᵢₙ₁ₛ-Ri plants. FIG. 10a: Schematic diagram showing the regions targeted by FTᵢₙ₁ₛ-Ri (top bar under diagram) and fragments of bisulfite sequenced (bottom bar under diagram). FIG. 10b: DNA methylation levels on the overlapping regions between regions targeted by FTᵢₙ₁ₛ-Ri and fragments of bisulfite sequenced in FTᵢₙ₁ₛ-Ri-2, FTᵢₙ₁ₛ-Ri-3 and EGFP-Ri. Data shown are means of two biological replicates±SEM. FIG. 10c: DNA methylation patterns on the FT intronic regions of FTᵢₙ₁ₛ-Ri-2. The numbers on the X-axis refer to the nucleotide positions downstream of the FT intron 1 first 5′ nt. Left box indicates cytosines located in CArG boxes and right box indicates cytosines targeted by FTᵢₙ₁ₛ-Ri.

FIG. 11 shows DNA methylation patterns on the FT intronic regions of FTᵢₙ₁ₛ-Ri-2,17c-3 double mutant plants. The numbers on the X-axis refer to the nucleotide positions downstream of the FT intron 1 first 5′ nt. Left box indicates cytosines located in CArG boxes and right box indicates cytosines targeted by FTᵢₙ₁ₛ-Ri.

FIGS. 12a-12e show histone modifications on the FT locus. FIG. 12a: Schematic diagram showing the FT gene structure and primer pairs (P1 to P5) used for ChIP PCR. FIG. 12b: ChIP assay using anti-H3K9me2 antibody or IgG (negative control) were conducted with WT and FTᵢₙ₁ₛ-Ri-2 plants. Tai was used as a positive control. Enrichments compared to input DNA were shown. Three different activated lines (FTᵢₙ₁-Ri-1, FTᵢₙ₁-Ri-6, and FTᵢₙ₁ₛ-Ri-2), WT, and one EGFP RNAi control line (EGFPi) were analyzed using anti-H3K27me3 (FIG. 12c), H3K36me2 (FIGS. 12d), and H3K36me3 (FIG. 12e) antibody. For quantifications, the relative enrichments against input DNA were calculated, and relative enrichments are shown to those of the control, AGAMOUS (AG, c) or ACTT (FIGS. 12d and 12e). All quantitative PCR data shown are means±SD.

FIGS. 13a-13c show DNA methylation and histone modification on the FT locus in FTₚᵣₒ-Ri plants. FIG. 13a: Schematic diagram showing the FTₚᵣₒ-Ri targeted regions and primer pairs used for ChIP PCR (A to C and P2) and chop-PCR. FIG. 13b: DNA methylation on the FT promoter region was determined by chop-PCR. Genomic DNAs were digested by McrBC followed by PCR, and no McrBC digestion used as control. FIG. 13c: H3K9me3 modifications on the FT locus in Col-0, EGFP-Ri and two independent lines of FTₚᵣₒ-Ri plants. For quantifications, the relative enrichments against input DNA were calculated, and enrichments are shown relative to those of the control, Tai.

FIG. 14 shows organ expression pattern of FT in the indicated seedlings. Data shown are means of three biological replicates±SD.

### DETAILED DESCRIPTION OF THE INVENTION

The present invention relates to transcriptional gene activation. More specifically, the present invention relates to the methylation of the gene body using inverted repeat RNAs which results in the activation of gene expression without changing the tempo-spatial regulatory patterns of the gene expression.

As described and shown herein, using the FT locus as a model gene, evidence is provided that gene body DNA methylation facilitated transcription by blocking binding of a negative transcription factor and/or altering chromatin modification in Arabidopsis. More importantly, the tempo-spatial expression patterns of FT were maintained in the activated lines. A similar result was also obtained for other Arabidopsis genes.

Unless defined otherwise, all technical and scientific terms used herein have the same meaning as is commonly understood by one of skill in the art to which the invention belongs.

The term “about” or “approximately” means within a statistically meaningful range of a value. Such a range can be within an order of magnitude, preferably within 50%, more preferably within 20%, more preferably still within 10%, and even more preferably within 5% of a given value or range. The allowable variation encompassed by the term “about” or “approximately” depends on the particular system under study, and can be readily appreciated by one of ordinary skill in the art.

The term “activation,” as it refers to genes activated by the subject RNAi method, refers to an increase in the level of expression of a gene(s) in the presence of one or more RNAi construct(s) when compared to the level in the absence of such RNAi construct(s). The term “activation” is used herein to indicate that the target gene expression is increased by about 5 times to about 10 times. For example, the expression may be raised by about 5 times, 5.5 times, 6 times, 6.5 times, 7 times, 7.5 times, 8 times, 8.5 times, 9 times, 9.5 times or 10 times. The term “activation” is used herein further to indicate that the target gene expression is increased by more than 5 times, more than 5.5 times, more than 6.0 times, more than 6.5 times, preferably more than 7 times, more than 7.5 times, more than 8.0 times, more than 8.5 times, or most preferably more than 9 times, more than 9.5 times, or more than 10.0 times.

A “dsRNA” or “dsRNA molecule” or “RNAi molecule” as used herein in the context of RNAi, refers to a compound, which is capable of activating or increasing the expression of a gene or the activity of the product of such gene to an extent sufficient to achieve a desired biological or physiological effect or to confer a phentotype in a plant. A dsRNA may be a hairpin construct that comprises a loop or spacer sequence which joins the two strands of the double stranded portion of the hairpin construct. The loop or spacer may be derived from cleavage of an intron during transcription. A dsRNA may also be a loopless hairpin construct.

The term “expression” with respect to a gene sequence refers to transcription of the gene and, as appropriate, translation of the resulting mRNA transcript to a protein. Thus, as will be clear from the context, expression of an RNA results from transcription of a polynucleotide. Similarly as will be clear from the context, expression of a protein results from transcription and translation of a polynucleotide.

As used herein, “gene” refers to a nucleic acid sequence that encompasses a 5′ promoter region associated with the expression of the gene product, any intron and exon regions and 3′ or 5′ untranslated regions associated with the expression of the gene product.

The term “gene activation” refers to the increase in gene expression, e.g., transgene, heterologous gene and/or endogenous gene expression. Gene activation may be mediated through processes that affect methylation of introns of the gene. Gene activation may be allele-specific wherein specific activation of one allele of a gene occurs.

The term “heterologous” or “exogenous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous or exogenous protein indicates that the protein comprises two or more subsequences that are not found in the same relationship to each other in nature (e.g., a fusion protein).

As used herein, the term “nucleic acid fragment” refers to a polynucleotide that comprises a portion of a larger polynucleotide. Typically, a nucleic acid fragment comprises a sequence of nucleotides of at least about 100 nucleotides in length, at least about 150 nucleotides in length, at least about 200 nucleotides in length, at least about 250 nucleotides in length, at least about 300 nucleotides in length, at least about 350 nucleotides in length, at least about 400 nucleotides in length, at least about 450 nucleotides in length, at least about 500 nucleotides in length, at least about 550 nucleotides in length, at least about 600 nucleotides in length, at least about 650 nucleotides in length, at least about 700 nucleotides in length, at least about 750 nucleotides in length, at least about 800 nucleotides in length, at least about 850 nucleotides in length, at least about 900 nucleotides in length, at least about 950 nucleotides in length, or at least about 1000 nucleotides in length. Typically, a nucleic acid fragment comprises about 100 to about 1000 nucleotides, about 100 to about 1000 nucleotides, about 150 to about 1000 nucleotides, about 200 to about 1000 nucleotides, about 250 to about 1000 nucleotides, about 300 to about 1000 nucleotides, about 350 to about 1000 nucleotides, about 400 to about 1000 nucleotides, about 450 to about 1000 nucleotides, about 500 to about 1000 nucleotides, about 550 to about 1000 nucleotides, about 600 to about 1000 nucleotides, about 650 to about 1000 nucleotides, about 700 to about 1000 nucleotides, about 750 to about 1000 nucleotides, about 800 to about 1000 nucleotides, about 850 to about 1000 nucleotides, about 900 to about 1000 nucleotides, or about 950 to about 1000 nucleotides. The larger polynucleotide may be a plant gene, preferably a plant gene which confers a phenotype.

“Operable linkage” or “operably linked” as used herein is understood as meaning, for example, the sequential arrangement of a promoter and the nucleic acid to be expressed and, if appropriate, further regulatory elements such as, for example, a terminator, in such a way that each of the regulatory elements can fulfill its function in the recombinant expression of the nucleic acid to make dsRNA. This does not necessarily require direct linkage in the chemical sense. Genetic control sequences such as, for example, enhancer sequences, can also exert their function on the target sequence from positions which are somewhat distant, or indeed from other DNA molecules (cis or trans localization). Preferred arrangements are those in which the nucleic acid sequence to be expressed recombinantly is positioned downstream of the sequence which acts as promoter, so that the two sequences are covalently bonded with one another.

As used herein, “phenotype” refers to the detectable characteristics of a cell or organism, which characteristics are the manifestation of gene expression. In a plant, desired phenotypes may be improved resistance against fungal, bacterial, viral, or animal pathogens, improved tolerance against abiotic stress(es) like light stress, heat stress, drought stress, soil salinity, heavy metals, water excess, cold stress, frost stress and pollutants such as ozone, enhanced seed yield or biomass yield, improved quality with respect to ingredients in any tissues, ability to confer sterility or restauration of sterility, altered lodging, or enhanced herbicide resistance.

The term “plant” according to the present invention includes whole plants or parts of such a whole plant, preferably an eudicot, dicot and monocot plant. The plant according to the present invention may be selected from the group consisting of barley (Hordeum vulgare), sorghum (Sorghum bicolor), rye (Secale cereale), Triticale, sugar cane (Saccharum officinarium), maize (Zea mays), foxtail millet (Setaria italic), rice (Oryza sativa), Oryza minuta, Oryza australiensis, Oryza alta, wheat (Triticum aestivum), Triticum durum, Hordeum bulbosum, purple false brome (Brachypodium distachyon), sea barley (Hordeum marinum), goat grass (Aegilops tauschii), apple (Malus domestica), Beta vulgaris, sunflower (Helianthus annuus), Australian carrot (Daucus glochidiatus), American wild carrot (Daucus pusillus), Daucus muricatus, carrot (Daucus carota), eucalyptus (Eucalyptus grandis), Erythranthe guttata, Genlisea aurea, woodland tobacco (Nicotiana sylvestris), tobacco (Nicotiana tabacum), Nicotiana tomentosiformis, tomato (Solanum lycopersicum), potato (Solanum tuberosum), coffee (Coffea canephora), grape vine (Vitis vinifera), cucumber (Cucumis sativus), mulberry (Morus notabilis), thale cress (Arabidopsis thaliana), Arabidopsis lyrata, sand rock-cress (Arabidopsis arenosa), Crucihimalaya himalaica, Crucihimalaya wallichii, wavy bittercress (Cardamine flexuosa), peppergrass (Lepidium virginicum), sheperd's-purse (Capsella bursa-pastoris), Olmarabidopsis pumila, hairy rockcress (Arabis hirsuta), rape (Brassica napus), broccoli (Brassica oleracea), Brassica rapa, Brassica juncacea, black mustard (Brassica nigra), radish (Raphanus sativus), Eruca vesicaria sativa, orange (Citrus sinensis), Jatropha curcas, Glycine max, and black cottonwood (Populus trichocarpa).

The terms “polynucleotide”, “nucleic acid” and “nucleic acid molecule” are used interchangeably herein to refer to a polymer of nucleotides which may be a natural or synthetic linear and sequential array of nucleotides and/or nucleosides, including deoxyribonucleic acid, ribonucleic acid, and derivatives thereof. It includes chromosomal DNA, self-replicating plasmids, infectious polymers of DNA or RNA and DNA or RNA that performs a primarily structural role. Unless otherwise indicated, nucleic acids or polynucleotide are written left to right in 5′ to 3′ orientation. Nucleotides are referred to by their commonly accepted single-letter codes. Numeric ranges are inclusive of the numbers defining the range. The “nucleic acid” may also optionally contain non-naturally occurring or altered nucleotide bases that permit correct read through by a polymerase and do not reduce expression of the nucleic acid.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers Amino acids may be referred to by their commonly known three-letter or one-letter symbols. Amino acid sequences are written left to right in amino to carboxy orientation, respectively. Numeric ranges are inclusive of the numbers defining the range.

As used herein, the term “substantially homologous” or “substantial homology”, with reference to a nucleic acid sequence, includes a nucleotide sequence that hybridizes under stringent conditions to a referenced SEQ ID NO:, or a portion or complement thereof, are those that allow an antiparallel alignment to take place between the two sequences, and the two sequences are then able, under stringent conditions, to form hydrogen bonds with corresponding bases on the opposite strand to form a duplex molecule that is sufficiently stable under conditions of appropriate stringency, including high stringency, to be detectable using methods well known in the art. Substantially homologous sequences may have from about 70% to about 80% sequence identity, or more preferably from about 80% to about 85% sequence identity, or most preferable from about 90% to about 95% sequence identity, to about 99% sequence identity, to the referent nucleotide sequences as set forth the sequence listing, or the complements thereof. Alternatively, substantially homologous sequences include those which hybridize under stringent conditions to the target regions of introns of plant genes. As used herein, stringent conditions are those that (1) employ low ionic strength and high temperature for washing, for example, 0.015 M NaCl/0.0015 M sodium citrate/0.1% NaDodSO₄ at 50° C.; (2) employ during hybridization a denaturing agent such as formamide, for example, 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, SxDenhardt's solution, sonicated salmon sperm DNA (50 g/ml), 0.1% SDS and 10% dextran sulfate at 42° C. in 0.2×SSC and 0.1% SDS. For stringency conditions, see also U.S. Pat. Nos. 8,455,716 and 8,536,403.

As used herein, the term “sequence identity”, “sequence similarity” or “homology” is used to describe sequence relationships between two or more nucleotide sequences. The percentage of “sequence identity” between two sequences is determined by comparing two optimally aligned sequences over a comparison window such as the full length of a referenced SEQ ID NO:, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. A sequence that is identical at every position in comparison to a reference sequence is said to be identical to the reference sequence and vice-versa. A first nucleotide sequence when observed in the 5′ to 3′ direction is said to be a “complement” of, or complementary to, a second or reference nucleotide sequence observed in the 3′ to 5′ direction if the first nucleotide sequence exhibits complete complementarity with the second or reference sequence. As used herein, nucleic acid sequence molecules are said to exhibit “complete complementarity” when every nucleotide of one of the sequences read 5′ to 3′ is complementary to every nucleotide of the other sequence when read 3′ to 5′. A nucleotide sequence that is complementary to a reference nucleotide sequence will exhibit a sequence identical to the reverse complement sequence of the reference nucleotide sequence. These terms and descriptions are well defined in the art and are easily understood by those of ordinary skill in the art.

As used herein, a “comparison window” or “window of comparison” refers to a conceptual segment of at least 6 contiguous positions, usually about 50 to about 100, more usually about 100 to about 150, in which a sequence is compared to a reference sequence of the same number of contiguous positions after the two sequences are optimally aligned. The comparison window may comprise additions or deletions (i.e. gaps) of about 20% or less as compared to the reference sequence (which does not comprise additions or deletions) for optimal alignment of the two sequences Those skilled in the art should refer to the detailed methods used for sequence alignment, such as in the Wisconsin Genetics Software Package Release 7.0 (Genetics Computer Group, 575 Science Drive Madison, Wis., USA).

As used herein, a “target gene” refers to a gene whose expression is to be activated. “A target region” refers to a genomic region comprising a target gene. Preferably the target gene or the target region confers a desired phenotype.

The present invention provides methods and compositions for activating expression of one or more genes in plants while maintaining the tempo-spatial regulatory patterns of the expression of the one or more genes. Preferably the tissue specificity and temporal expression pattern are maintained. The gene activation is based on gene body DNA methylation induced by dsRNA, dsRNA fusion constructs or hairpin constructs described herein. The modified methylation pattern can result in blocking binding of a negative transcription factor and/or altering chromatin modification in a plant. Alternatively, the modified methylation pattern can result only in altering chromatin modification without blocking binding of a negative transcription factor; that means that methylation occurs at a position in an intron which does not comprise a binding site of a negative transcription factor, but nevertheless, gene activation also occurs in this instance. In certain embodiments the gene activation is provided by expression of a dsRNA fusion construct. In some embodiments of the invention, the dsRNA interferes with expression of a target gene described herein. In one embodiment RNA-mediated gene activation can be conferred by the expression of an inverted-repeat transgene cassette that generates a population of small interfering RNAs (siRNAs) (also sometimes referred to herein as small RNAs (smRNA)) derived from the dsRNA region of a transgene transcript.

Thus in one aspect, the present invention provides a nucleic acid construct which comprises one or more nucleic acid fragments. In one embodiment, the nucleic acid construct comprises one nucleic acid fragment which comprises a nucleotide sequence substantially homologous to a DNA target region within an intron of a target gene in a plant for activating the target gene or the expression of the target gene. In another embodiment, the nucleic acid construct comprises two nucleic acid fragments in which each nucleic acid fragment comprises a nucleotide sequence substantially homologous to a target region within the same intron of a target gene for activating the target gene such that the expression of the target gene is activated. In another embodiment, the nucleic acid construct comprises two nucleic acid fragments in which each nucleic acid fragment comprises a nucleotide sequence substantially homologous to a target region within different introns of the same target gene for activating the target gene such that the expression of the target gene is activated. In another embodiment, the nucleic acid construct comprises two nucleic acid fragments in which each nucleic acid fragment comprises a nucleotide sequence substantially homologous to a target region within an intron of a target gene for activating the target gene such that the expression of two different target genes is activated. In a further embodiment, the nucleic acid construct comprises three nucleic acid fragments in which each nucleic acid comprises a nucleotide sequence substantially homologous to a target region within an intron of a target gene for activating the target gene such that the expression of three target genes is activated. In some embodiments, two or more nucleic acid fragments are linked together in the nucleic acid construct. In other embodiments where the construct comprises two or more nucleic acid fragments, each nucleic acid fragment comprises a nucleotide sequence substantially homologous to target regions within introns of one, two or more target genes for activating the one, two or more target genes.

In some embodiments, the one or more nucleic acid fragments are present in the nucleic acid construct such that a dsRNA is produced and the dsRNA comprises a nucleotide sequence substantially homologous to a target region of at least one nucleic acid fragment of the one or more nucleic acid fragments or a part of said nucleotide sequence. Preferably, the dsRNA comprises nucleotide sequences substantially homologous to target region(s) of all nucleic acid fragments of the one or more nucleic acid fragments or parts of said nucleotide sequences. In other embodiments, the one or more nucleic acid fragments are present in the nucleic acid construct such that a hairpin construct is produced upon expression of the one or more nucleic acid fragments and the double stranded portion of the hairpin construct comprises a nucleotide sequence substantially homologous to a target region of at least one nucleic acid fragment of the one or more nucleic acid fragments or a part of said nucleotide sequence. Preferably, the double stranded portion of the hairpin construct comprises nucleotide sequences substantially homologous to target region(s) of all nucleic acid fragments of the one or more nucleic acid fragments or parts of said nucleotide sequences. In one embodiment, the nucleic acid construct comprises one or more nucleic acid fragments in a sense orientation with respect to the promoter, a spacer sequence that can form a loop and the one or more nucleic acid fragments in an antisense orientation with respect to the promoter, wherein the one or more nucleic acid fragments in antisense orientation are preferably in reverse order with respect to the order of the one or more nucleic acid fragments in sense orientation. In another embodiment, the nucleic acid construct comprises one or more nucleic acid fragments in an antisense orientation with respect to the promoter, a spacer sequence that can form a loop and the one or more nucleic acid fragments in a sense orientation with respect to the promoter, wherein the one or more nucleic acid fragments in sense orientation are preferably in reverse order with respect to the order of the one or more nucleic acid fragments in antisense orientation. The spacer sequence may comprise, for example, a sequence of nucleotides of at least about 10-100 nucleotides in length, or alternatively at least about 100-200 nucleotides in length, at least 200-400 about nucleotides in length, at least about 400-500 nucleotides in length, or more than about 500 nucleotides. In an additional embodiment, the nucleic acid construct comprises one or more nucleic acid fragments in a sense orientation with respect to the promoter, an intron and the one or more nucleic acid fragments in an antisense orientation with respect to the promoter, wherein the one or more nucleic acid fragments in antisense orientation are preferably in reverse order with respect to the order of the one or more nucleic acid fragments in sense orientation. In a further embodiment, the nucleic acid construct comprises one or more nucleic acid fragments in an antisense orientation with respect to the promoter, an intron and the one or more nucleic acid fragments in a sense orientation with respect to the promoter, wherein the one or more nucleic acid fragments in sense orientation are preferably in reverse order with respect to the order of the one or more nucleic acid fragments in antisense orientation. In some embodiments, the spacer or the intron is removed from the nucleic acid construct during transcription to produce a loop-less hairpin (Smith et al., 2000).

RNAi molecules, particularly dsRNA molecules described herein, can be prepared by the skilled artisan using techniques well known in the art, including techniques for the selection and testing of RNAi molecules that are useful for activating gene expression as described herein. See, for example, Wesley et al. (2001), Kalantidis et al. (2002), Mysara et al. (2011), Yan et al. (2012) and Qu et al. (2012). It has typically been found that dsRNA of 200-700 by are particularly suited for inducing RNAi in plants. It has also been found that hairpin RNAs containing an intron, for example, a construct comprising an RNA encoding sequence in a sense direction operably linked to an intron operably linked to an RNA encoding sequence in an antisense direction or vice versa which is capable of forming an intron-hairpin RNA (ihpRNA), is suitable for inducing RNAi in plants. See, for example, Wang et al. (2000), Fuentes et al. (2006), Bonfim et al. (2007) Vanderschuren et al. (2007a, 2007b), Zrachya et al. (2007). For example, a nucleic acid construct can be prepared that includes a nucleic acid that is transcribed into an RNA that can anneal to itself, e.g., a double stranded RNA having a stem-loop structure. In addition, hairpin structures can be prepared as described by Guo et al. (2003).

The size of the nucleic acid construct is selected so that it is capable of forming dsRNA or a hairpin construct that is cleaved to form small interfering (si) RNAs and the formed dsRNA or hairpin construct targets one, two or more genes for increasing expression of the target genes as described herein. In some embodiments, the dsRNA is a hairpin having a loop. In other embodiments, the dsRNA is a loop-less hairpin. In one embodiment, the length of the double stranded portion of the dsRNA comprises more than about 100 bp, more than about 150 bp, more than about 200 bp, more than about 250 bp, more than about 300 bp, more than about 350 bp, more than about 400 bp, more than about 450 bp, more than about 500 bp, more than about 550 bp, more than about 600 bp, more than about 650 bp, more than about 700 bp, more than about 750 bp, more than about 800 bp, more than about 850 bp, more than about 900 bp, more than about 950 bp, more than about 1000 bp, more than about 1050 bp, or more than about 1100 bp. In another embodiment, the length of the double stranded portion of the dsRNA comprises about 100 by to about 1200 bp. In a further embodiment, the length of the double stranded portion of the dsRNA comprises about 150 by to about 1200 bp. In a further embodiment, the length of the double stranded portion of the dsRNA comprises about 200 by to about 1200 bp. In an additional embodiment, the length of the double stranded portion of the dsRNA comprises about 250 by to about 1200 bp. In another embodiment, the length of the double stranded portion of the dsRNA comprises about 300 by to about 1200 bp. In a further embodiment, the length of the double stranded portion of the dsRNA comprises about 350 by to about 1200 bp. In an additional embodiment, the length of the double stranded portion of the dsRNA comprises about 400 by to about 1200 bp. In another embodiment, the length of the double stranded portion of the dsRNA comprises about 450 by to about 1200 bp. In a further embodiment, the length of the double stranded portion of the dsRNA comprises about 500 by to about 1200 bp. In a further embodiment, the length of the double stranded portion of the dsRNA comprises about 550 by to about 1200 bp. In an additional embodiment, the length of the double stranded portion of the dsRNA comprises about 600 by to about 1200 bp. In another embodiment, the length of the double stranded portion of the dsRNA comprises about 650 by to about 1200 bp. In a further embodiment, the length of the double stranded portion of the dsRNA comprises about 700 by to about 1200 bp. In an additional embodiment, the length of the double stranded portion of the dsRNA comprises about 750 by to about 1200 bp.

In another embodiment, the length of the double stranded portion of the dsRNA comprises about 800 by to about 1200 bp. In a further embodiment, the length of the double stranded portion of the dsRNA comprises about 850 by to about 1200 bp. In an additional embodiment, the length of the double stranded portion of the dsRNA comprises about 900 by to about 1200 bp. In another embodiment, the length of the double stranded portion of the dsRNA comprises about 950 by to about 1200 bp. In an additional embodiment, the length of the double stranded portion of the dsRNA comprises about 1000 by to about 1200 bp. In another embodiment, the length of the double stranded portion of the dsRNA comprises about 1050 by to about 1200 bp. In a further embodiment, the length of the double stranded portion of the dsRNA comprises about 1100 by to about 1200 bp. In another embodiment, the length of the double stranded portion of the dsRNA comprises about 1150 by to about 1200 bp. In a further embodiment, the length of the double stranded portion of the dsRNA comprises about 200 by to about 300 bp. In an additional embodiment, the length of the double stranded portion of the dsRNA comprises about 500 by to about 550 bp. in a further embodiment, the length of the double stranded portion of the dsRNA comprises about 550 by to about 600 bp. In another embodiment, the length of the double stranded portion of the dsRNA comprises about 600 by to about 650 bp. In one embodiment, the length of the double stranded portion of the dsRNA comprises about 250 bp. In another embodiment, the length of the double stranded portion of the dsRNA comprises about 500 bp. In a further embodiment, the length of the double stranded portion of the dsRNA comprises about 550 bp. In a further embodiment, the length of the double stranded portion of the dsRNA comprises about 600 bp. In another embodiment, the length of the double stranded portion of the dsRNA is 1000 bp.

Although specific nucleic acid constructs and nucleic acid fragments are shown in the Examples, it is understood that these constructs are illustrative only and that other constructs and fragments can be prepared using the techniques described herein. Such nucleic acid constructs and nucleic acid fragments are prepared so that they or parts thereof are substantially homologous to a nucleotide sequence within target regions of introns of plant genes. The plant genes may be transgenes or endogens, preferably endogens. Other nucleic acid constructs include those with variations of the target region. In one embodiment, a variation of the target region is one in which the target region is shifted 5′ of a disclosed or designed target region of a gene described herein. In another embodiment, a variation of the target region is one in which the target region is shifted 3′ of a disclosed or designed target region of a gene described herein. In an additional embodiment, the variation of a target region can be sequence a variation for more effective activation of genes described herein or homologs thereof.

In some embodiments, nucleic acid fragments of the present invention include those which hybridize under stringent conditions to the target regions of introns of plant genes. As used herein, stringent conditions are those that (1) employ low ionic strength and high temperature for washing, for example, 0.015 M NaCl/0.0015 M sodium citrate/0.1% NaDodSO₄ at 50° C.; (2) employ during hybridization a denaturing agent such as formamide, for example, 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 g/ml), 0.1% SDS and 10% dextran sulfate at 42° C. in 0.2×SSC and 0.1% SDS. For stringency conditions, see also U.S. Patent Nos. 8,455,716 and 8,536,403.

In some embodiments, the nucleic acid construct further comprises a plant operable promoter operably linked to a nucleic acid fragment. In one embodiment, a plant operable promoter is operably linked to each nucleic acid fragment. In another embodiment, a plant operable promoter is operably linked to two or more nucleic acid fragments that area linked together. In some embodiments, the plant operable promoter is heterologous to the one or more nucleic acid fragments or to polynucleotide(s) from which the one or more nucleic acid fragments are portions or to the target gene(s) to which the nucleotide sequence(s) within the one or more nucleic acid fragments are substantially homologous to. In some embodiments, the plant operable promoter is a promoter of a polynucleotide(s) from which one or more nucleic acid fragments derived or a promoter of a target gene(s) to which the nucleotide sequence(s) within one or more nucleic acid fragments are substantially homologous to. In a preferred embodiment, a heterologous promoter exhibits the same or a comparable specificity/inducibility like the promoter of a polynucleotide(s) from which one or more nucleic acid fragments derived or a promoter of a target gene(s) to which the nucleotide sequence(s) within one or more nucleic acid fragments are substantially homologous to. Additionally, a desired specificity/inducibility of a promoter can be achieved by use of chimeric/synthetic promoters. Such promoters can be designed according to the desired requirements and are induced or repressed by various factors. Chimeric/synthetic promoters do not occur in nature and are assembled from multiple elements. Typically, they comprise a minimal promoter as well as, upstream of the minimal promoter, at least one cis-regulatory element, which serves as the bonding location for specific transcription factors. In some embodiments, the nucleic acid construct further comprises a plant operable terminator. In one embodiment, a plant operable terminator is operably linked to each nucleic acid fragment. In another embodiment, a plant operable terminator is operably linked to two or more nucleic acid fragments that area linked together. In some embodiments, the plant operable terminator is heterologous to the one or more nucleic acid fragments or to polynucleotide(s) from which the one or more nucleic acid fragments are portions or to the target gene(s) to which the nucleotide sequence(s) within the one or more nucleic acid fragments are substantially homologous to. In some embodiments, the plant operable terminator is a terminator of a polynucleotide(s) from which one or more nucleic acid fragments derived or a terminator of a target gene(s) to which the nucleotide sequence(s) within one or more nucleic acid fragments are substantially homologous to.

A number of promoters can be used in the practice of the invention. The promoters can be selected based on the desired outcome. That is, the nucleic acid fragments can be combined with constitutive, tissue-preferred, or other promoters for expression in the host cell of interest. Such constitutive promoters include, for example, the core promoter of the Rsyn7 (WO 99/48338 and U.S. Patent No. 6,072,050); the core CaMV 35S promoter (Odell et al., 1985; U.S. Patent No. 5,850,019); rice actin (McElroy et al., 1990); ubiquitin (Christensen and Quail, 1989; Christensen et al., 1992); pEMU (Last et al., 1991); MAS (Velten et al., 1984); ALS promoter (U.S. Pat. No. 5,659,026), and the like. Other constitutive promoters include, for example, those disclosed in U.S. Pat. Nos. 5,608,149; 5,608,144; 5,604,121; 5,569,597; 5,466,785; 5,399,680; 5,268,463; 5,608,142 and 8,455,716. In some embodiments, the promoter is a duplicated CaMV 35S promoter.

Other promoters include inducible promoters, particularly from a pathogen-inducible promoter. Such promoters include those from pathogenesis-related proteins (PR proteins), which are induced following contact or infection by a pathogen; e.g., PR proteins, SAR proteins, beta-1,3-glucanase, chitinase, etc. Other promoters include those that are induced locally at or near the site of pathogen infection. In further embodiments, the promoter may be a wound-inducible promoter. In further embodiments, the promoter may be a promoter responsive to abiotic stress like light stress, heat stress, drought stress, soil salinity, heavy metals, water excess, cold stress, frost stress and pollutants such as ozone. In other embodiments, chemical-regulated promoters can be used to modulate the expression of one or more nucleic acid fragments in a plant through the application of an exogenous chemical regulator. The promoter may be a chemical-inducible promoter, where application of the chemical inducesexpression, or a chemical-repressible promoter, where application of the chemical represses expression. In addition, tissue-preferred promoters can be utilized to target expression of one or more nucleic acid fragments within a particular plant tissue. Furthermore, developmental promoters can be utilized to target expression during particular developmental stages like germination, early development, flowering, bolting, maturity or ripening. Exemplary, some of these promoters are described in U.S. Pat. Nos. 6,506,962, 6,575,814, 6,972,349 and 7,301,069 and in U.S. Patent Application Publication Nos. 2007/0061917 and 2007/0143880.

In some embodiments, plant operable terminators include those from the nopaline synthase gene of A. tumefaciens (NOS), octopine synthase gene of A. tumefaciens (OCS), the terminator for the T7 transcript from the octopine synthase gene of A. tumefaciens, and the pea RUBISCO synthase E9 gene (E9 3′) 3′ non-translated transcription termination and polyadenylation sequence. These and other terminators and 3′ end regulatory sequences are well known in the art. See, e.g., U.S. Pat. Nos. 8,344,209, 8,373,022 and 8,569,583.

A nucleic acid construct that comprises a plant operable promoter or a plant operable promoter and a plant operable terminator may also be referred to herein as an expression cassette. The expression cassette may include other transcriptional regulatory regions as are well known in the art.

In other embodiments, the nucleic acid construct or expression cassette further comprises a selectable marker gene. Selectable marker genes are utilized for the selection of transformed cells or tissues. Typically, a selectable marker gene is located downstream of the nucleic acid construct which produces the dsRNA molecule(s) or the hairpin construct(s), so that the expression of the selectable marker gene is an indicator for the complete introduction of the nucleic acid construct or expression cassette into the host cell. Usually, the plant selectable marker gene will encode antibiotic resistance, with suitable genes including at least one set of genes coding for resistance to the antibiotic spectinomycin, the streptomycin phosphotransferase (spt) gene coding for streptomycin resistance, the neomycin phosphotransferase (nptll) gene encoding kanamycin or geneticin resistance, the hygromycin phosphotransferase (hpt or aphiv) gene encoding resistance to hygromycin, acetolactate synthase (als) genes. Alternatively, the plant selectable marker gene will encode herbicide resistance such as resistance to the sulfonylurea-type herbicides, glufosinate, glyphosate, ammonium, bromoxynil, imidazolinones, and 2,4-dichlorophenoxyacetate (2,4-D), including genes coding for resistance to herbicides which act to inhibit the action of glutamine synthase such as phosphinothricin or basta (e.g., the bar gene). See generally, International Publication No. WO 02/36782, U.S. Pat. No. 7,205,453 and U.S. Patent Application Publication Nos. 2006/0218670, 2006/0248616, 2007/0143880 and 2009/0100536, and the references cited therein. See also, Jefferson et al. (1991); De Wet et al. (1987); Goff et al. (1990); Kain et al. (1995) and Chiu et al. (1996). This list of selectable marker genes is not meant to be limiting. Any selectable marker gene can be used. The selectable marker gene is also under control of a promoter operable in the plant species to be transformed. Such promoters include those described in International Publication No. WO 2008/094127 and the references cited therein.

In other embodiments, the nucleic acid construct or expression cassette further comprises a selectable marker. In some embodiments, the selectable marker is part of a recombination marker free system. In one embodiment, the recombination marker free system is a Cre-lox recombination marker free system, such as described by Zuo et al. (2001), a zinc finger marker free system, a TALE nucleases marker free system or a CRISPR-Cas marker free system, each of which are well known in the art. In some embodiments, the recombination marker free system is positioned between the plant operable promoter and the one or more nucleic acid fragments.

In preparing the nucleic acid construct or an expression cassette, the various DNA elements like promoters, terminators, introns, nucleic acid fragments etc. may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g. transitions and transversions may be involved.

Nucleic acid constructs or expression cassettes of the present invention may also be synthesized, either completely or in part, especially where it is desirable to provide plant-preferred sequences, by methods known in the art.

In a second aspect, the present invention provides a transgenic plant comprising one or more nucleic acid constructs described herein in which of the target gene(s) is/are activated in the transgenic plant. In some embodiments, the present invention provides any generation of plants having activated gene expression, including those described herein. In other embodiments, the present invention provides a transgenic plant seed of any generation of such plants. In some embodiments, the present invention provides a method to prepare transgenic plants and seeds described herein.

In a further aspect, the present invention provides an expression vector comprsing the nucleic acid construct or the expression cassette of the present invention. The expression vector may bemay be introduced into a plant cell using conventional transformation procedures. The term “plant cell” is intended to encompass any cell derived from a plant including undifferentiated tissues such as callus and suspension cultures, as well as plant seeds, pollen or plant embryos. Plant tissues suitable for transformation include leaf tissues, root tissues, meristems, protoplasts, hypocotyls, cotyledons, scutellum, shoot apex, root, immature embryo, pollen, and anther. “Transformation” means the directed modification of the genome of a cell by the external application of recombinant DNA from another cell of different genotype, leading to its uptake and integration into the subject cell's genome. In this manner, genetically modified plants, plant cells, plant tissue, seed, and the like can be obtained.

DNA or nucleic acid constructs described herein can be used to transform any plant. The constructs may be introduced into the genome of the desired host by a variety of conventional techniques. Techniques for transforming a wide variety of higher plant species are well known and described in the technical and scientific literature. Transformation protocols may vary depending on the type of plant or plant cell, i.e., monocot or dicot, targeted for transformation, as is well known to the skilled artisan. For example, the DNA construct may be introduced directly into the genomic DNA of the plant cell using techniques such as electroporation and microinjection of plant cell protoplasts, or the DNA constructs can be introduced directly to plant tissue using ballistic methods, such as DNA particle bombardment. Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. Thus, any method, which provides for effective transformation/transfection may be employed. See, for example, U.S. Pat. Nos. 7,241,937, 7,273,966 and 7,291,765 and U.S. Patent Application Publication Nos. 2007/0231905 and 2008/0010704 and references cited therein. See also, International Publication Nos. WO 2005/103271 and WO 2008/094127 and references cited therein.

Transformed plant cells which are derived by any of the above transformation techniques can be cultured to regenerate a whole plant which possesses the transformed genotype and thus the desired phenotype, e.g., a transgenic plant. A “transgenic plant” is a plant into which foreign DNA has been introduced. A “transgenic plant” encompasses all descendants, hybrids, and crosses thereof, whether reproduced sexually or asexually, and which continue to harbor the foreign DNA. Regeneration techniques rely on manipulation of certain phytohormones in a tissue culture growth medium, typically relying on a biocide and/or herbicide marker which has been introduced together with the desired nucleotide sequences. See for example, International Publication No. WO 2008/094127 and references cited therein. See also, U.S. Patent Application Publication Nos. 2010/0304488, 2011/0117652, 2011/0247099, 2012/0073018, 2012/0246759 and 2012/0272403.

The foregoing methods for transformation are typically used for producing a transgenic plant in which the nucleic acid construct or expression cassette is stably incorporated into the genome. The nucleic acid construct or nucleic acid fragment for gene activation from transgenic plants produced in accordance with the present invention can be transferred to other plants by sexual crossing. In one embodiment, the transgenic plant could be crossed with another (non-transformed or transformed) plant in order to produce a new transgenic plant having the nucleic acid construct or nucleic acid fragment for gene activation. Alternatively, the nucleic acid construct or nucleic acid fragment for gene activation could be moved into another plant line using traditional backcrossing techniques that are well known in the plant breeding arts. For example, a backcrossing approach could be used to move the nucleic acid construct or nucleic acid fragment for gene activation from a non-elite germplasm into an elite germplasm, or from a germplasm containing another foreign undesired gene in its genome into a germplasm which do not contain that gene. As used herein, “crossing” can refer to a simple X by Y cross, or the process of backcrossing, depending on the context. Any of a number of standard breeding techniques can be used, depending upon the species to be crossed.

The transgenic plants produced in accordance with the present invention can be cultivated in accordance with conventional procedures. Transgenic seeds can, of course, be recovered from the transgenic plants. These seeds can then be planted in the soil and cultivated using conventional procedures to produce transgenic plants.

In one embodiment, the transformation of the nucleic acid construct or the expression cassette or the expression vector is transient, i.e. in a method for transformation for producing or preparing a transgenic plant in which the nucleic acid construct or expression cassette is not stably incorporated into the genome. In that case, the production of dsRNA or the hairpin construct in the transiently transformed plant leads to gene body DNA methylation. Since the DNA methylation is a heritable modification, the achieved gene activation is maintained over several plant generations, even though the nucleic acid construct or the expression cassette or the expression vector is not further present in the transformed plant cell. In another embodiment, the gene body DNA methylation can be made by direct (external) application of smRNAs or siRNAs on a plant cell, plant tissue or plant. It is well-known in the art that smRNAs or siRNAs, can be introduced in a plant cell or the nucleus or a plastid or mitochondria of a plant cell, e.g. by means of a nuclear localization signal or a plastid signal sequence or the like or a mitochondrial targeting sequence or the like. This introduction can optionally be by means of a carrier, e.g., a particle used for particle bombardment or by polyethylene glycol or by salmon sperm or the like. These techniques and others are well known to the skilled artisan.

In a third aspect, the present invention provides a method for activating gene expression in a plant or a method of conferring increased target gene expression in a plant, preferably a transgenic plant, described herein, the method comprises expressing in the plant the nucleic acid construct or the expression cassette of the present invention. In some embodiments, the method comprises expressing in the plant at least one nucleic acid construct that expresses dsRNA targeting one or more introns of one or more target genes. In some embodiments, one, two or three nucleic acid constructs that express dsRNA targeting introns of target genes are used to confer activation of gene expression. The target genes can be transgenes or endogenes.

In a fourth aspect, the present invention provides a method of altering the phenotype of a transgenic plant. In accordance with this aspect, the method comprises stably incorporating in the genome of a plant a nucleic acid construct described herein that encodes a dsRNA molecule that when expressed in a transgenic plant activates or increases expression of a target gene which confers the altered phenotype of the transgenic plant. The altered phenotype may be characterized by plant morphology, physiology, growth and development, yield, nutritional enhancement, disease or pest resistance, or environmental or chemical tolerance. In some aspects, the altered phenotype is selected from group consisting of enhanced water use efficiency, enhanced temperature tolerance, increased yield, enhanced nitrogen use efficiency, enhanced seed protein, enhanced seed oil and enhanced biomass. Increase yield may include increased yield under non-stress conditions and increased yield under environmental stress conditions. Stress conditions may include, for example, drought, shade, fungal disease, viral disease, bacterial disease, insect infestation, nematode infestation, extreme temperature exposure (cold or hot), osmotic stress, reduced nitrogen nutrient availability, reduced phosphorus nutrient availability and high plant density.

In a fifth aspect, the present invention provides a method of preparing a non-transgenic plant exhibiting an increased expression or activation of an endogenous target gene and/or an altered phenotype. The increased expression or activation of the target gene can be determined using conventional molecular biology techniques well known to the skilled artisan. Alternatively, the increased expression or activation of a target gene or of an altered phenotype can be determined by observing the non-transgenic plants as is well known to the skilled artisan. In accordance with this aspect, the present invention further provides a plant prepared by this method. In one embodiment, the method comprises (a) stably incorporating in the genome of a plant a nucleic acid construct described herein that encodes a dsRNA molecule that when expressed in a transgenic plant activates expression of the endogenous target gene which confers the altered phenotype of the transgenic plant, (b) crossing the transgenic plant of step (a) with a non-transgenic plant to produce progeny, and (c) selecting progeny from step (b) in which the incorporated nucleic acid construct has been eliminated due to segregation and which exhibits an increased activation of an endogenous target gene and/or an altered phenotype. The elimination of the nucleic acid construct can be determined using conventional molecular biology techniques well known to the skilled artisan.

In another embodiment, the method comprises (a) transiently incorporating in plant cells a nucleic acid construct described herein that encodes a dsRNA molecule, (b) selecting a transgenic plant cell of step (a) in which the expression of the endogenous target gene is activated, (c) proliferating the plant cell of step (b) to produce a population of plant cells in which the expression of the endogenous target gene is activated, (d) selecting a plant cell from the population of plant cells of step (c) in which the expression of the endogenous target gene is activated and which does not contain the nucleic acid construct, and (e) regenerating a non-transgenic plant from the selected cell of step (d) in which the plant exhibits an increased activation of an endogenous target gene and/or an altered phenotype.

In a further embodiment, the method comprises (a) introducing siRNA or smRNA produced by a nucleic acid construct described herein into a plant cell, (b) selecting a plant cell of step (a) in which the expression of the endogenous target gene is activated, and (c) regenerating a non-transgenic plant from the selected cell of step (b), in which the plant exhibits an increased activation of an endogenous target gene and/or an altered phenotype.

Overexpression is an important strategy to increase production of a desired protein, as well as to study gene function, especially when a knock-out mutant is not available. However, the significance of overexpression phenotype is largely dependent on the promoter used to transcribe the target gene. The widely used cauliflower mosaic virus 35S promoter as well as other constitutive promoter used in plant studies elevated transgene expression but compromised the tempo-spatial expression patterns of the native gene (Benfy and Chua, 1990). Overexpression of a gene product in inappropriate cell types may lead to artifacts or toxic lethality. The present invention demonstrates activation of, i.e., increased, gene expression without introducing an extra-copy of the target gene; most importantly, the tempo-spatial expression of targeted gene was maintained. Gene expression is activated by targeting an intron of a target gene. This strategy can be used not only to increase expression of transgenes and endogenes in plants, but also to study gene function by tissue-specific biotechnology applications of overexpression.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, New York); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Green and Sambrook, 2012, Molecular Cloning, 4th Ed. (Cold Spring Harbor Laboratory Press, Cold Spring Harbor, N.Y.); Ausubel et al., 1992, Current Protocols in Molecular Biology (John Wiley & Sons, including periodic updates); Glover, 1985, DNA Cloning (IRL Press, Oxford); Russell, 1984, Molecular biology of plants: a laboratory course manual (Cold Spring Harbor Laboratory Press, Cold Spring Harbor, N.Y.); Anand, Techniques for the Analysis of Complex Genomes, (Academic Press, New York, 1992); Guthrie and Fink, Guide to Yeast Genetics and Molecular Biology (Academic Press, New York, 1991); Harlow and Lane, 1988, Antibodies, (Cold Spring Harbor Laboratory Press, Cold Spring Harbor, N.Y.); Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Fire et al., RNA Interference Technology: From Basic Science to Drug Development, Cambridge University Press, Cambridge, 2005; Schepers, RNA Interference in Practice, Wiley—VCH, 2005; Engelke, RNA Interference (RNAi): The Nuts & Bolts of siRNA Technology, DNA Press, 2003; Gott, RNA Interference, Editing, and Modification: Methods and Protocols (Methods in Molecular Biology), Human Press, Totowa, N.J., 2004; Sohail, Gene Silencing by RNA Interference: Technology and Application, CRC, 2004.

### EXAMPLES

The present invention is described by reference to the following Examples, which is offered by way of illustration and is not intended to limit the invention in any manner. Standard techniques well known in the art or the techniques specifically described below were utilized.

### Example 1

### Materials and Methods

Genes: Seed of various Arabidopsis mutants were provided by Dr. Hervé Vaucheret, Dr. Pablo Vera, Dr. Steven E. Jacobsen, Dr. Xiaofeng Cao and Dr. Richard M. Amasino.

The Arabidopsis Genome Initiative locus number for the genes used in these examples are: At1g18100 for MFT, At1g65480 for FT, At1g69120 for API, At2g38940 for ATPT2, At2g45660 for SOCl, At3g07650 for COLS, At3g60140 for DIN2, At4g15210 for BMY1, At4g20370 for TSF, At4g24540 for AGL24, At5g03840 for TFL1, At5g10140 for FLC, At5g15840 for CO, and At5g61850 for LFY.

Construction: Using standard PCR technique, the following fragments of the Arabidopsis FT locus (translation start site as +1): FTₚᵣₒ (−746 to −129) (SEQ ID N0:69), FTᵢₙ₁ (+202 to +1015) (SEQ ID NO:70), FTᵢₙ₁ₛ (+334 to +900) (SEQ ID NO:71, FTᵢₙ₂ (+1078 to +1790) (SEQ ID NO:72) were amplified from Arabidopsis genomic DNA and FT, (+78 to +511) (SEQ ID NO:73) was amplified from a cDNA library by primer pairs listed in Table 1. After digestion and purification, the fragments were cloned into pENTR3C vector (Invitrogen). RNAi constructs were generated by LR reaction using Gateway LR Clonase (Invitrogen) with pH7GWIWG2(II) (Karimi et al., 2002). Using the same strategy, RNAi constructs targeting genomic fragment -1007 to -624 of ATPT2 (SEQ ID NO:74) and +212 to +615 of BMY1 (SEQ ID NO:75) (refer to translation start site as +1) were generated. 35S promoter of pH7GWIWG2(II) was replaced with a ˜1.2 kb UBQ10 promoter to generate a RNAi vector transcribed from the UBQ10 promoter. To generate gFT-GUS construct, a ˜7.8 kb genomic DNA including the entire FT coding region and ˜5.7 kb promoter region was amplified using gFT-F and gFT-R primers. The fragment was then cloned into pCambia-1391z (CAMBIA Australia).

TABLE 1
Oligonucleotides

| DNA or cDNA cloning | Sequence | (SEQ ID NO:) |
|---|---|---|
| pFTi_BamHF | TGAAGGATCCCTTGTTCTACCTG (1) | |
| pFT_NotR | TTCGCGGCCGCATTAACTCGGGTCGGTGAAAT (2) | |
| FTex_F | CACGTCGACTCTAAAGGTTACTTATGGCCAAAGAGAG (3) | |
| FTex_R | TTCGCGGCCGCAGCCACTCTCCCTCTGACAAT (4) | |
| FTin1_F | CACCGGTACCGTTCTTTCACTTGAACTCCCTT (5) | |
| FTin1_R | GTCGACTCGAGCTTCATTAAACAAATAAGACGA (6) | |
| FLC_BamHF | CACCGGATCCATGGGAAGAAAAAAACTAGAAATCA (7) | |
| FLC_NotR | TTCGCGGCCGCGAATTAAGTAGTGGGAGAGTCACCG (8) | |
| pUBQ10_F | TGCAAGCTTCCTAGGACCGGATAAGTTCCCTTC (9) | |
| pUBQ10_R | TACTAGTCTCGAGCTGTTAATCAGAAAAACTCAGATTAATCG (10) | |
| FTin1s_F | CACCGGTACCTTGGTTAAAAATGCCCCACGC (11) | |
| FTin1s_R | GTCGACTCGAGATAACACAAGAAAGAAGAA (12) | |
| FTin2_F | CACCGGTACCGTTTGTGCACTAACTCAACTCT (13) | |
| FTin2_R | GTCGACTCGAGCTGTAATGAGAGATAGCCATAC (14) | |
| gFT_F | TATCTCGACTCCATAATTTCTAACGAACATTTGC (76) | |
| gFT_R | TATGCGGCCGCAGTCTTCTTCCTCCGCAGCCAC(77) | |
| ATPT2i_BamHF | CACCGGATCCCTGTTCAATCTCTAATTCGTC (15) | |
| ATPT2i_NotR | TTCGCGGCCGCGATTACTATTTGTCTTCAGTTACCCA (16) | |
| BMY1i_SalF | CACCGTCGACCCTTTAATGGCCCCATTAGT (17) | |
| BMY1i_NotR | TTCGCGGCCGCATTGATAGTTATGATCAAAGACATTGTT (18) | |
| qRT-PCR | Sequence | (SEQ ID NO:) |
| AP1_RtF | TGGTCTGTATCAAGAAGATGATCCTATGGC (19) | |
| AP1_RtR | ATGGAAATGCTTCATGCGGCGAA (20) | |
| MFT_RtF | AGCAGCCTCCATCACGAGCCAAT (21) | |
| MFT_RtR | CAGGTTCCTTTTGGGCGTTGAAAT (22) | |
| LFY_RtF | TGGCTCATGGCGTCAGGCTTGTT (23) | |
| LFY_RtR | CCAAATAGAGAGACGAGGATGAGCGT (24) | |
| TFL1_RtF | AGAGATCACTTCAACACTCGTAAATTTGCG (25) | |
| TFL1_RtR | TGCGTGCAGCGGTTTCTCTTTGT (26) | |
| TSF_RtF | CGTATTGTGTTGGTATTGTTCCGGC (27) | |
| TSF_RtR | GGAAGACCAAGATTGTAGATCTCAGCAAAC (28) | |
| SOC1_RtF | TGGGGATCTCATGAAAGCGAAGTTT (29) | |
| SOC1_RtR | CCCAATGAACAATTGCGTCTCTACTTCA (30) | |
| BMY1_RtF | GCAGCGAAGTACGGGCATGAGAT (31) | |
| BMY1_RtR | CCCACTTAAATGCTCCACTTGGC (32) | |
| BMY2_RtF | ATGCGCATGAAGAAGCATTAGCTGATCC (33) | |
| BMY2_RtR | CAAGTAATCGCATTCTCTCCACCGA (34) | |
| ACT2_RtF | CGCCATCCAAGCTGTTCTC (35) | |
| ACT2_RtR | CAAGACGGAGGATGGCATGAG (36) | |
| FT_RtF | TTTCTACAATTGTCAGAGGGAGAGTGG (37) | |
| FT_RtR | CATCACCGTTCGTTACTCGTATCAT (38) | |
| AGL24_RTF | GACTTAGCCGTGTGTCTGAAAAGA (39) | |
| AGL24_RTR | AGCCTCTTTAAGCGTCGTCAGT (40) | |
| GUS_RtF | CTGCATCAGCCGATTATCATCACC (78) | |
| GUS_RtR | CATACCTGTTCACCGACGACGG (79) | |
| APTP1_RtF | TGATGATCTTGTGCTCTGTCG (80) | |
| ATPT1_RtR | ATGACACCCTTGGCTTCG (81) | |
| FLC_RtF | TGTGAGTATCGATGCTCTTGTTCA (41) | |
| FLC_RtR | TTCAACATGAGTTCGGTCTTCTTG (42) | |
| CO_RtF | GGTGATAAGGATGCCAAGGAGGTTG (43) | |
| CO_RtR | GTCCATACTCGAGTTGTAATCCACAAGG (44) | |
| ATPT2_RtF | AGGAGCAATGGTTGGTGCGTTCG (45) | |
| ATPT2_RtR | CAATAAGCGAGTTCCTGACCCCA (46) | |
| Bisulfite sequencing | Sequence | (SEQ ID NO:) |
| FT_BiF1 | TGGTGGAGAAGAYYTYAGGAA (47) | |
| FT_BiR1 | AATCCCAARATTCTTATCCTAAATAA (48) | |
| ChIP-qPCR | Sequence | (SEQ ID NO:) |
| FT_ChIP1F | GTGGCTACCAAGTGGGAGAT (49) | |
| FT_ChIP1R | TAACTCGGGTCGGTGAAATC(50) | |
| FT_ChIP2F | CCAAGAGTTGAGATTGGTGGA (51) | |
| FT_ChIP2R | GGGCATTTTTAACCAAGGTCT (52) | |
| FT_ChIP3F | TCAAGCCAGCCTTTAAGATACTCTCTGC (53) | |
| FT_ChIP3R | GGGTTGCTAGGACTTGGAACATCTG (54) | |
| FT_ChIP4F | GCTCAAACATGTTGCTCGAA (55) | |
| FT_ChIP4R | TGCGATCAGTAAAATACACAGACA (56) | |
| FT_ChIP5F | GATCTACAATCTCGGCCTTCC (57) | |
| FT_ChIP5R | ATCATCACCGTTCGTTACTCG (58) | |
| FT_ChIPAF | TTCCTTTATTTTCCAGTTTGGACAG (82) | |
| FT_ChIPAR | TTGCACGACCAGGATAATTG (83) | |
| FT_ChIPBF | GTTTGTCGACCATATAACACAAGCGG (84) | |
| FT_ChIPBR | CGAAAGCGAAAACGTTCTAAAAGAAAA (85) | |
| FT_ChIPCF | AAGATTGTGGTTATGATTTCACCGACC (86) | |
| FT_ChIPCR | AACAAACAGGTGGTTTCTCTGTGTTGA (87) | |
| SOC1_ChIPF | TGGGAGGGAAAAAGATGTGT (59) | |
| SOC1_ChIPR | TGGTAATGGTGTTTGTGAAACC (60) | |
| AG_ChIPF | GTGAAACAAATTTTCCTGCAGAATGTCACT (61) | |
| AG_ChIPR | AGTTTTTGAGGCACTAAAATCTTTGGGTAAATC (62) | |
| Ta3_ChIPF | GATTCTTACTGTAAAGAACATGGCATTGAGAGA (63) | |
| Ta3_ChIPR | GATCCAAATTTCCTGAGGTGCTTGTAACC (64) | |
| Act7_chipF1 | CGTTTCGCTTTCCTTAGTGTTAGCT (65) | |
| Act7_chipR1 | AGCGAACGGATCTAGAGACTCAC (66) | |
| genotyping | Sequence | (SEQ ID NO:) |
| ago4-2F | ATTGTTACTCAATGCATGGCTCC (67) | |
| ago4-2R | GCCATCGTCTTCAGTTCCATT(68) | |
| flc-3F | CACGTGGCAATCTTGTCTTCA (88) | |
| flc-3R | AGACGACGAGAAGAGCGACGG (89) | |

Plant material and growth conditions: Arabidopsis thaliana Columbia (Col-0) ecotype was used as the wild type (WT) control in this study. All A. thaliana mutants have been described: ago1 -27 (Morel et al., 2002), ago4-2 (Agorio and Vera, 2007), flc-3 (Michaels and Amasino, 1999), ddc (Zhang et al., 2006). All mutants are in the Columbia (Col-0) ecotype background. Plants were grown in a growth chamber under long-day (16 hour of light/8 hour of dark) condition illuminated by cool-white fluorescent light (100 μmol/m2/s). Transgenic plants were obtained by standard floral dip method (Clough and Bent, 1998). Double mutants were generated by genetic crosses. The mutation of ago4-2 and flc-3 were characterized by sequencing of the PCR product derived from the relevant genomic loci. ago1 -27 mutant and transgene were scored by phenotype and antibiotic tolerance, respectively. All samples were collected at the middle of the day (ZT, 8h) unless specified otherwise.

Phenotype observation: T1 transformants were selected on half strength MS medium plates supplemented with the appropriate antibiotic and positive plants were transferred to soil. Nine plants were planted for each 8×8 cm square plastic pot. To score the flowering time of T3 homozygous plants, seeds were geminated on half strength MS medium plate for 10-12 days and transferred to soil. At least 20 plants of each genotype were examined. To obtain double mutants, seedlings of F2 or F3 progeny descended from the same cross were chosen for experiments.

Gus staining: Two week old seedlings grown on half strengthen MS medium were transferred to staining buffer (0.1 M NaHPO₄ pH 7.0, 10 mM EDTA, 0.5 mM Potassium Ferrocyanide, 0.5 mM Potassium Ferricyanide, 0.1% Triton X-100, 0.5 mg/ml X-Gluc). After brief vacuum infiltration, the reaction mix was incubated at 37° C. overnight in darkness. GUS signals were examined under a microscope after destaining with 75% ethanol.

RNA extraction and real time quantitative PCR: RNA was extracted from 2-week old seedlings by RNeasy Plant Mini Kit (Qiagen) following the manufacturer's instructions. cDNA synthesis was performed by using Superscript III First strand synthesis system (Invitrogen) following the manufacturer's instructions. Real-time PCR was performed using SYBR Premix Ex Taq (Takara) in a Biorad CFX96 realtime PCR system using ACTIN2 as an internal control. The primers that were used are listed in Table 1. Real-time quantitative PCR was repeated with 3 biological replicates, and each sample was assayed in triplicate by PCR.

Small RNA Northern: RNA was extracted from 2-week old seedlings using TRIZOL reagent (Invitrogen) following the manufacturer's instructions. 20 μg total RNA per lane were used for gel blots. After separation on 15% PAGE/8 M Urea/1.0 X TBE buffer, the RNA was electroblotted to Hybond N⁺ membrane (Amersham, NJ) and then UV cross-linked. Probes were made by Prime-It II Random Primer Labeling Kits (Agilent Technologies) and purified using mini Quick Spin Columns (Roche) according to the manufacturer's instructions. Hybridizations were performed at 42° C. overnight in Oligo-UltraHyb hybridization solution (Ambion, TX), according to the supplier's direction. After hybridization, membranes were washed in 2X SSC with 0.1% SDS and analyzed using BioMax MS films (Kodak).

DNA methylation analysis: Arabidopsis genomic DNA was extracted from 2-week-old seedlings by DNeasy Plant Mini Kit (Qiagen). Bisulfite DNA conversion was performed by using 1 μg genomic DNA and EpiTech Bisulfite Kit (Qiagen) following the manufacturer's protocol. PCR was performed using primers located outside of the targeted region and designed for single strand methylation detection. PCR products were then cloned into the pGEM-T-Easy vector (Promega). For each genotype, at least 15 independent clones were sequenced using SP6 primer and the data were analyzed by Cymate (Hetzl et al., 2007). Bisulfite treatment was conducted twice with different patch of plant materials. For chop-PCR analysis of DNA methylation, 50 ng total DNAs were digested by McrBC (NEB) followed by PCR. McrBC specifically cleaves methylated DNA sequences.

Chromatin immunoprecipitation (ChIP) and ChIP-qPCR: ChIP assays were conducted as described previously (Deng et al., 2014). Antibodies to detect histone H3 trimethyl Lys4 (H3K4me3), H3K9me2, H3K9me3, H3K27me3, and H3K36me3 were from Active Motif (Cat.No. 39159, 39239, 39161, 39155, and 61101). Histone H3 Acetylation (H3K9/14ace) and histone H3 dimethyl Lys36 (H3K36me2) antibodies were from Milipore (Cat.No. 06-599 and 07-274). HA-antibody was obtained from Santa Cruz (sc-7392). The immunoprecipitates were analyzed by quantitative PCR as before. For H3K4me3, H3K36me2/3 and H3K9/14ace, ACTIN7 was used as an internal control. Tai and AGAMOUS were selected as a control for H3K9me2/3 and H3K27me3, respectively. The primers that were used are listed in Table 1.

### Example 2

### Activation of FT Expression by FTᵢₙ₁-Ri

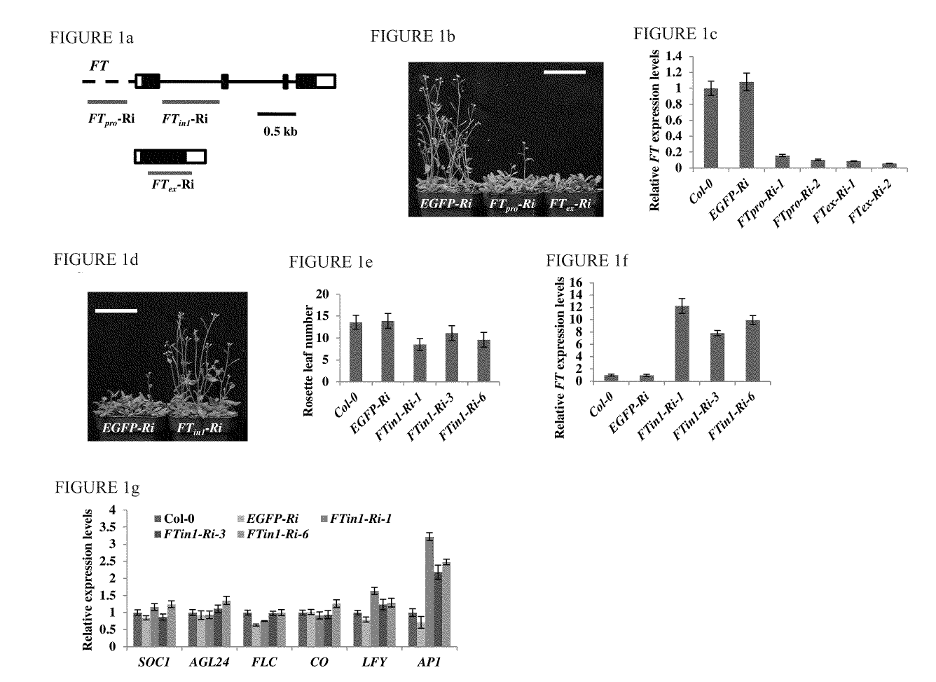

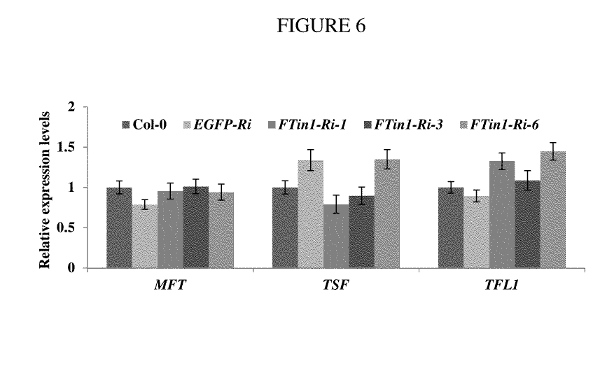

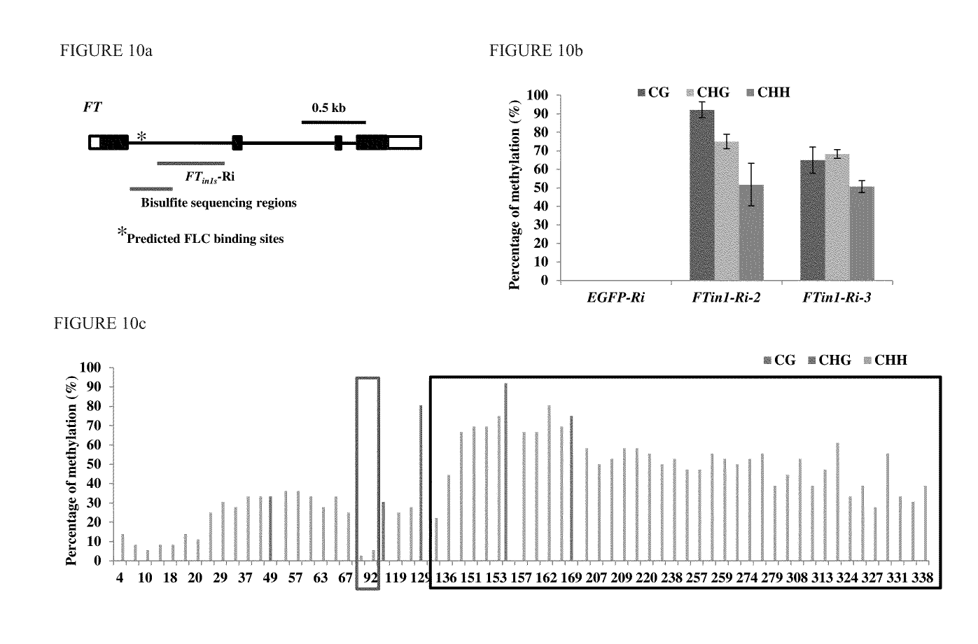

To investigate the function of DNA methylation in the regulating gene expression, several RNAi vectors (inverted-repeat RNAs, IRs) were constructed to target the FT promoter region (FTₚᵣₒ-Ri), exon regions (FTₚᵣₒ-Ri), and intronic regions (FTᵢₙ₁-Ri) (FIG. 1a). Most of the T1 transgenic plants expressing IR constructs targeting the promoter and exon of FT were found to be late flowering, in compared to EGFP RNAi control plants (FIG. 1b). Quantitative RT-PCR of two representative plants of each independent transgenic line showed the FT expression levels were were suppressed (FIG. 1c). However, FTᵢₙ₁-Ri T1 transgenic plants were early flowering (FIG. 1d) and FT transcript levels were higher in representative T3 homozygous plants (FIG. 10 whereas the mRNA levels of FT homologs TSF, MFT, and TFL1 were similar compared to WT and EGFP-Ri control plants (FIG. 6). The expression levels of upstream regulators of FT, CO and FLC, remained unchanged and the parallel pathway (SOCl and AGL24) was also unaffected. The direct downstream target of FT/FD complex, API was significantly activated, whereas the expression level of LEAFY, a direct target of SOCl/AGL24 was only slightly elevated (FIG. 1g). These data indicated that the FTᵢₙ₁-Ri construct specifically activated the endogenous FT, leading to early flowering of the transgenic plants.

### Example 3

### FTᵢₙ₁-Ri Activates FT Expression Through the RdDM Pathway

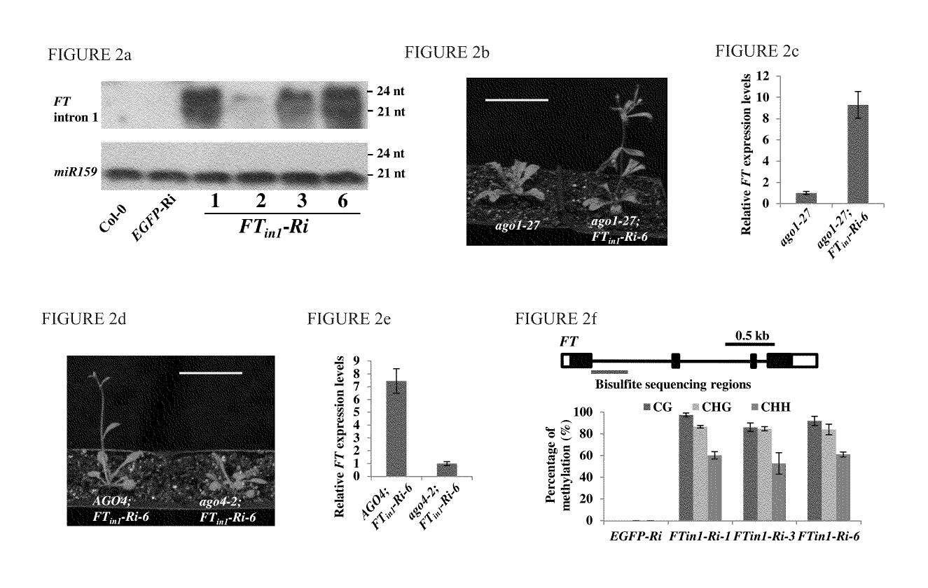

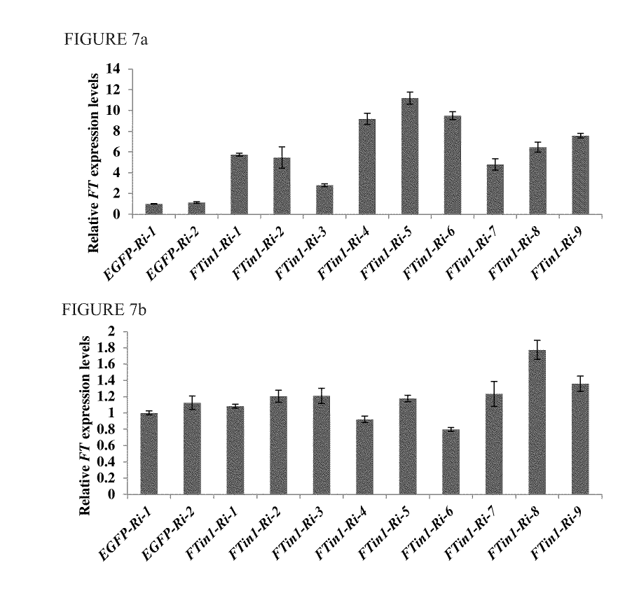

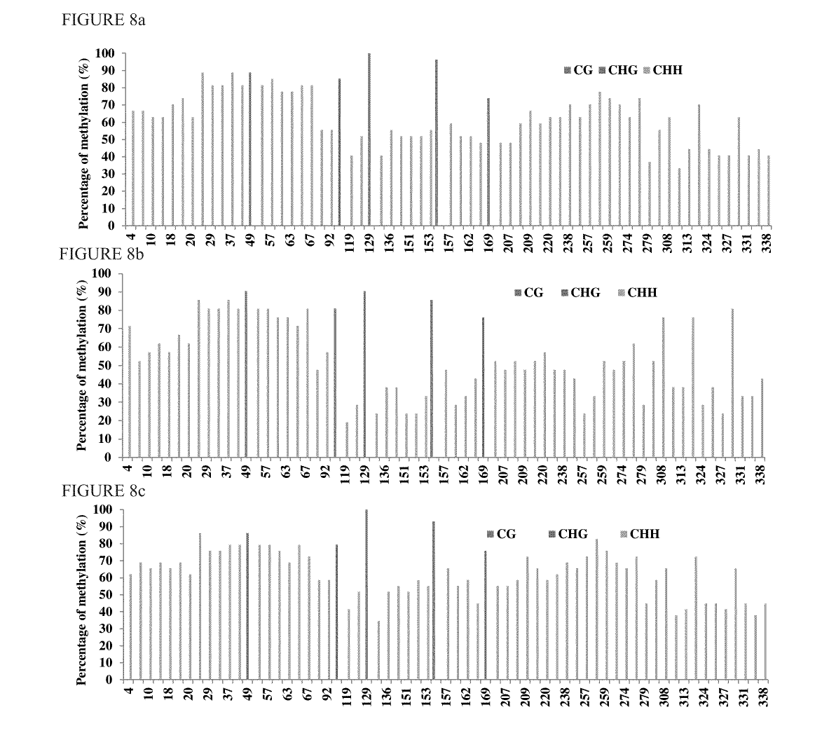

To investigate how FTᵢₙ₁-Ri activates FT expression, smRNA production by Northern blots was first examined. An abundance of ˜21-24 nt smRNAs was generated in FTᵢₙ₁-Ri transgenic plants, with ˜24 nt smRNAs as the major species (FIG. 2a). In Arabidopsis, 21 nt and 24 nt smRNAs are mainly loaded onto AGO1 and AGO4, respectively. To address which smRNA species was responsible for FT activation, an activated line, FTᵢₙ₁-Ri-6, was crossed with ago1 -27 and ago4-2, respectively. FIG. 2b shows ago1 -27/FTᵢₙ₁-Ri-6 homozygous plant still showed early flowering and the FT mRNA level was higher than in ago1-27 single mutant (FIG. 2c). By contrast, the FTᵢₙ₁-Ri-6 early flowering phenotype was reversed by ago4 mutation (FIG. 2d), and in FTᵢₙₛ-Ri-6;ago4-2 plants, FT mRNA expression was restored to WT levels (FIG. 2e). To further confirm the RdDM pathway was necessary for the activation, the same IR constructs but transcribed from a UBQ10 promoter were generated and WT and DNA methyltransferases mutant ddc (drml/drm2/cmt3) (Zhang et al., 2006) were transformed. Again, FT was activated in WT, but not in the RdDM pathway mutant ddc (FIGS. 7a and 7b). Sequencing of bisulfite-treated DNA showed that the endogenous FT intronic regions that were targeted by FTᵢₙ₁-Ri were heavy methylated (FIG. 2f; FIGS. 8a-8c). Together all the data indicated that the FTᵢₙ₁-Ri activated FT expression through RdDM pathway, independent of PTGS pathway.

### Example 4

### DNA Methylation of CArG Boxes Blocks FLC Binding to FT But Not Necessary for the Activation by the Intronic RNAi of FT

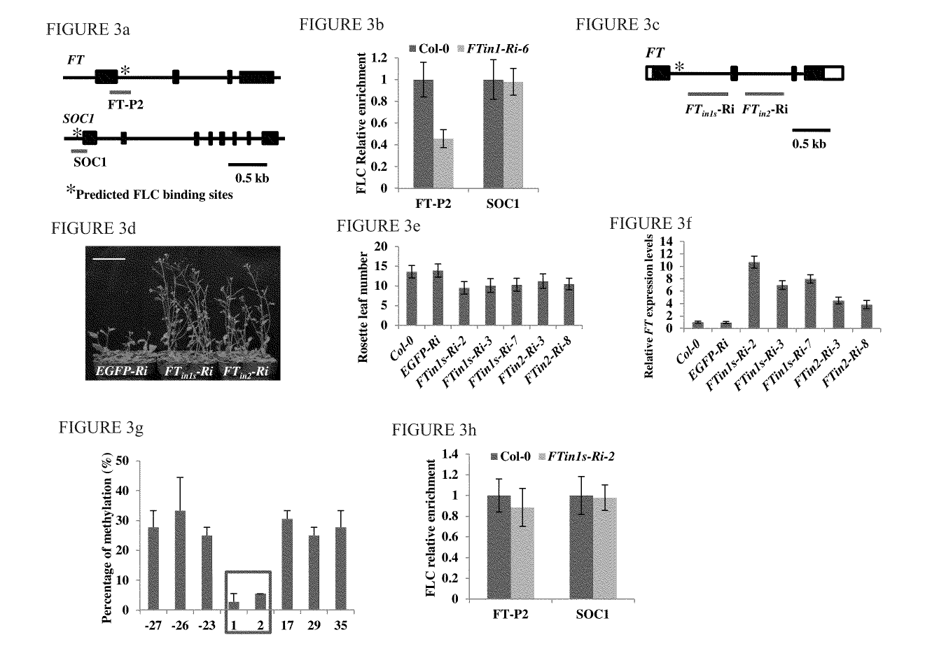

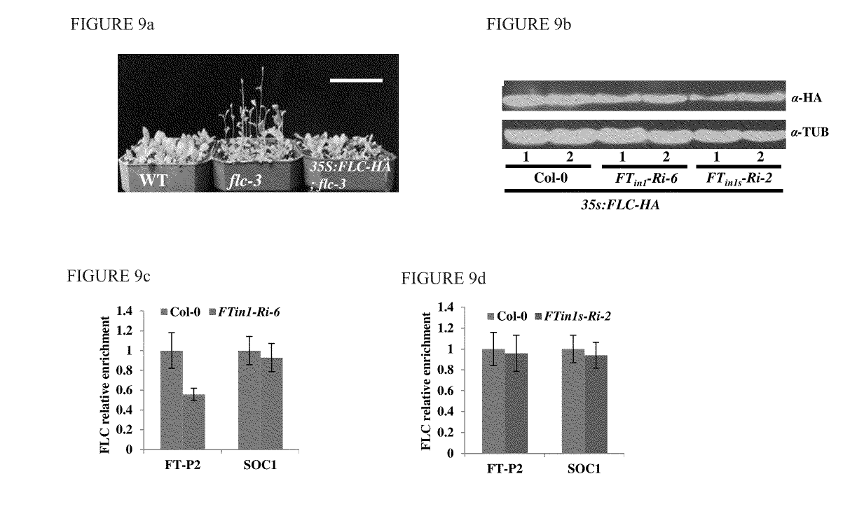

A previous study has demonstrated that the MADS transcription factor FLC directly binds to the CArG boxes of FT and SOCl to suppress their expression (Helliwell et al., 2006). To test whether the methylation would affect FLC binding to this region of the FT, 35S HA-tagged FLC fusion gene (35S:FLC-HA) was introduced into WT and FTᵢₙ₁-Ri-6 plants (FIGS. 9a-9d). This construct can complement the early flowering phenotype of the flc-3 indicating that the fusion protein was functional (FIGS. 9a-9d). Chromatin immunoprecipitation (ChIP) experiments showed that the FLC accumulation in the FT CArG region was reduced in FTᵢₙ₁-Ri-6 plants compared to WT plants. By contrast, FLC binding to the SOC1 CArG region remained unchanged in plants of both genotypes (FIGS. 3a and 3b and FIGS. 9a-9d).

Although the methylation of the CArG boxes in the first intron of FT blocked the accumulation of FLC on this region, some FTᵢₙ₁-Ri lines were also found to flower even earlier than the FLC knock out mutant flc-3. This may indicate that blocking of FLC binding was not the only mechanism to activate FT expression by FTᵢₙ₁-Ri. To obtain additional evidence, another two constructs targeting FT intronic regions were generated. FTᵢₙ₁ₛ-Ri, which targeted FT intron 1 but excluding the FLC binding sites and FTᵢₙ₂-Ri, which targeted FT intron 2 (FIG. 3c). T1 transgenic plants of both constructs flowered early in comparison with control EGFP-Ri transgenic plants (FIGS. 3d) and the FT expression levels were higher in T3 lines (FIG. 3f).

In Arabidopsis, the DNA methylation could spread to contiguous DNA region through a secondary RdDM pathway (Daxinger et al., 2009). To examine whether the CArG boxes were methylated by this mechanism, bisulfite sequencing was conducted to check the methylation status of these regions (FIG. 10a). Regardless of high methylation of the targeted regions by FTᵢₙ₁ₛ-Ri (FIG. 10b), clear DNA methylation spreading were detected in FTᵢₙ₁ₛ-Ri-2 (FIG. 10c). However, the methylation levels of the two cytosines within CArG boxes were significant lower compared with nearby cytosines (FIG. 3g). This indicated that FLC binding may have prevented the methylation machinery to target these two cytosines. To confirm this observation, 35S:FLC-HA was introduced into FTᵢₙ₁ₛ-Ri-2. FIG. 3h shows that FLC accumulation on FT was similar in both WT and FTᵢₙ₁ₛ-Ri-2 plants. However, the hypo-methylation status of CArG sites was only slightly recovered in FTᵢₙ₁ₛ-Ri-21flc-3 double mutants (FIG. 11). It is possible other MADS transcription factor(s) may bind to this region in the flc-3 mutant background.

### Example 5

### Histone Modification on the FT Locus

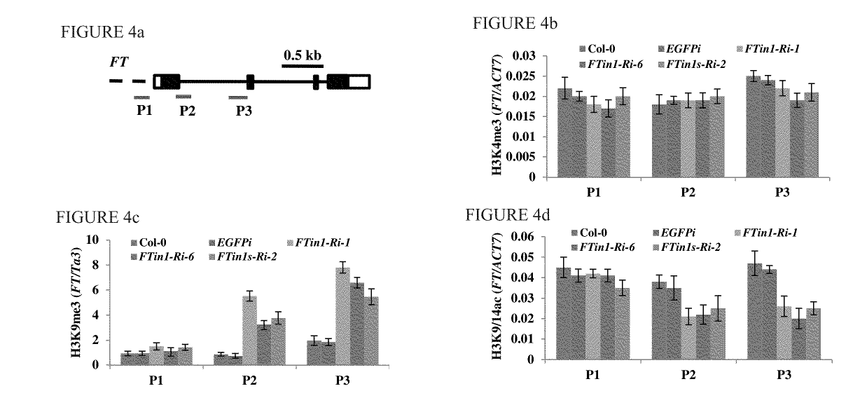

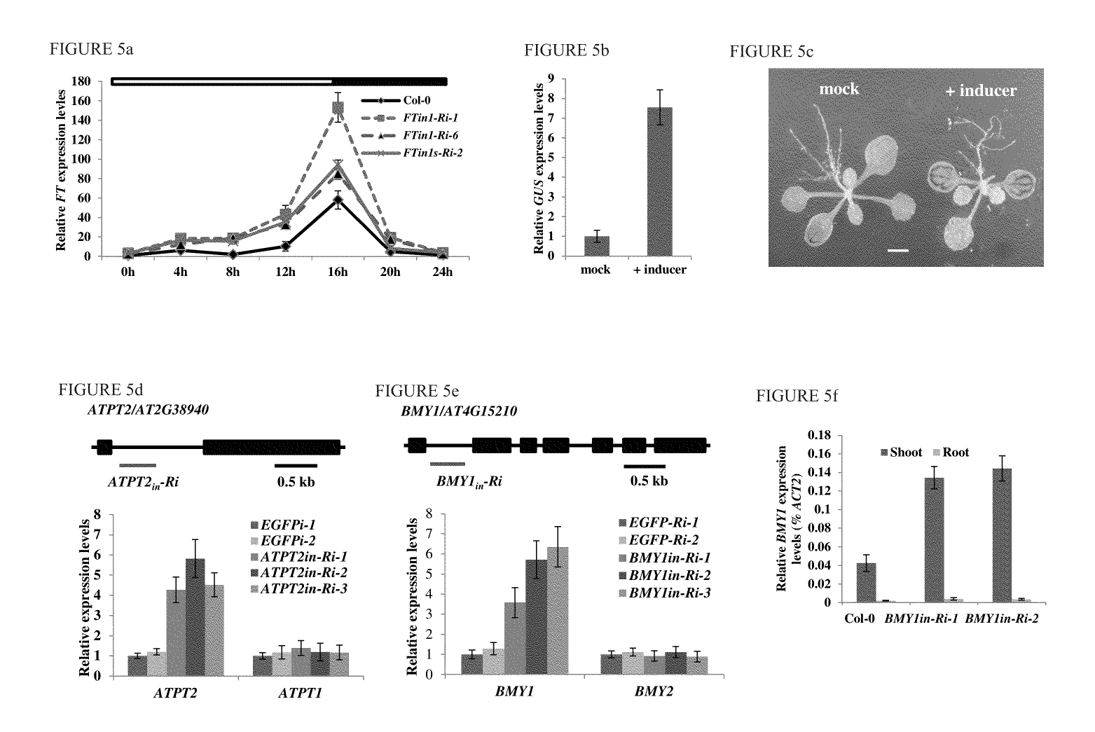

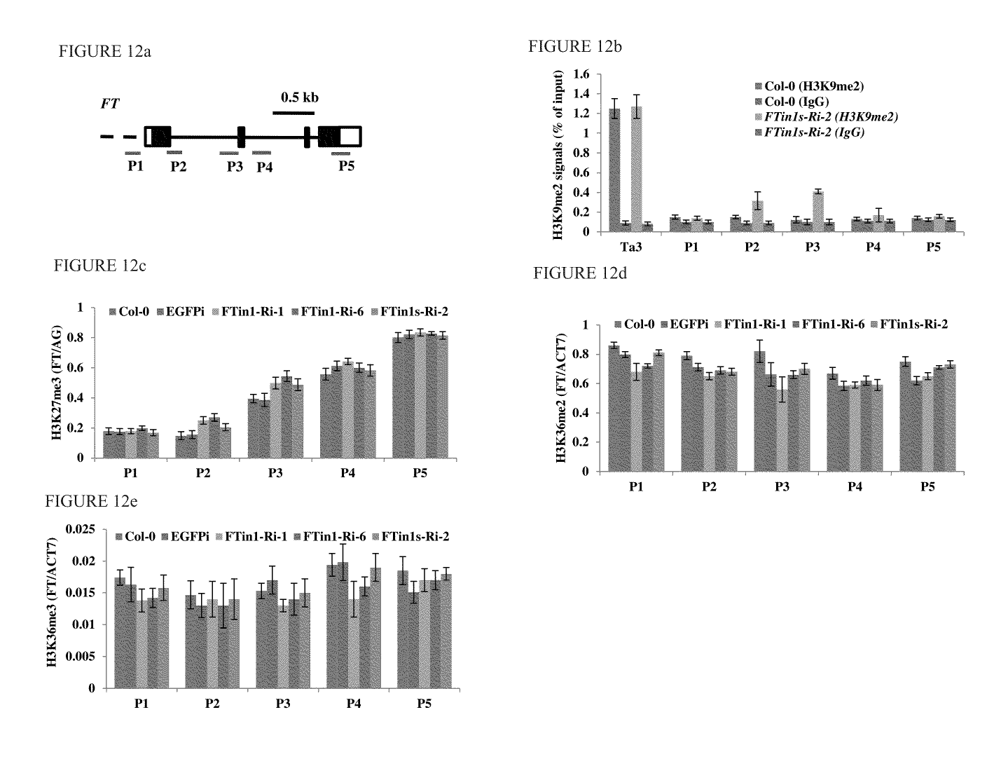

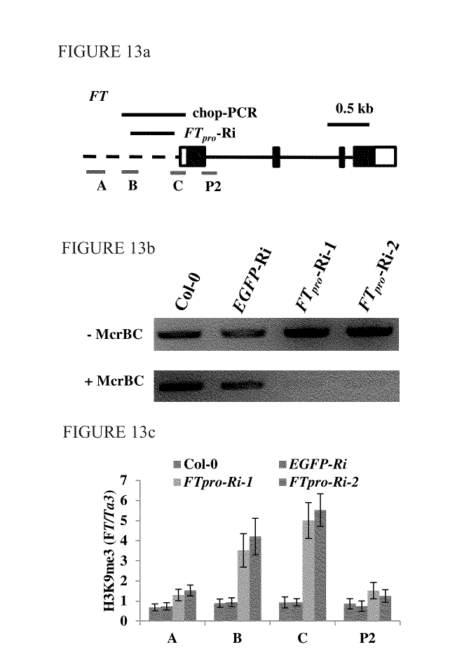

Other than affecting transcription factor binding affinity, DNA methylation may also regulate gene expression by altering chromatin structures (Razin, 1998). ChIP experiments were performed to examine histone modification status of the FT locus (FIG. 4a) in control and activated plants. No significant changes in H3K4me3 were detected among samples (FIG. 4b). Although H3K9me2, which is a typical histone marker accompanying DNA methylation, was hardly detectable on FT locus (FIG. 12b), association of H3K9me3 with FT was found. Furthermore, there was a significant increase of H3K9me3 around the targeted regions in activated plants (FIG. 4c). Correspondingly, the accumulation of H3K9/14 acetylation was reduced in these plants (FIG. 4d). A decrease of H3K27me3, a repression histone mark that has been reported to silence the FT locus (Adrian et al., 2010), was not detected in the activated plants (FIG. 12c). Gene body specific histone marker, H3K36me2/3, remained largely unchanged in all samples (FIGS. 12d and 12e). In FTₚᵣₒ-Ri plants, the FT promoter regions were heavily methylated and accompanied by high levels of H3K9me3 (FIGS. 13a-13c). FT expression was silenced in these plants (FIGS. 1b and 1c). The data indicated that promoter DNA methylation and H3K9me3 silence gene expression, whereas gene body DNA methylation and H3K9me3 promoter gene expression of FT.

### Example 6

### Maintenance of Target Gene Tempo-Spatial Expression Patterns Gained by Intronic RNAi

Time course detection of FT mRNA levels indicated the activated lines showed the same expression patterns as WT control (FIG. 5a). FT mRNA was hardly detected in roots even in the activated plants (FIG. 14]). To obtain further information on tissue-specific activation of FT by intronic RNAi, gFT-GUS;XVE:FTᵢₙ₁-Ri plants were generated, in which GUS encoding DNA were fused to a ˜8.7kb FT genomic DNA fragment and IRs targeting FT intron 1 was transcribed from a inducible promoter (Zuo et al., 2000). The transgene was activated when IRs were transcribed (+inducer) (FIG. 5b). Weak GUS signals were detected in leaf vascular tissue in 2-week old mock-treated seedlings. However, strong GUS signals were detected in the inducer treated seedlings (FIG. 5c). The GUS expression levels in distal part of leaves were higher than in proximal part and no expression was found in roots of both mock- and inducer-treated plants. These data indicated that the tempo-spatial expression patterns of targeted genes were maintained in plants activated by the intronic RNAi.

Using the same strategy, two other Arabidopsis genes, ATPT2/Pht1;4 and BMY1/RAM1, were specifically activated by RNAi constructs targeting the intronic regions of ATPT2 and BMY1, respectively (FIGS. 5d and 5e). The expression ratio of BMY1 between shoots and roots was found to be similar in the activation lines compared to wild type (FIG. 50.

### Example 7

### Discussion

The RdDM pathway has long been regarded as a repression machinery to silence transposons and repeat DNA sequences, as well as some protein coding genes (Matzke et al., 2009; Law and Jacobsen, 2010). The above Examples show that gene body DNA methylation introduced via RdDM pathway, in fact, activated FT mRNA expression.

DNA methylation may control transcription through the action of methyl-cytosine binding protein(s) or directly affect transcription factor(s) binding (Kass et al., 1997). However recently, there is still no direct evidence linking transcription factor binding affinity to DNA methylation status in plants. Furthermore, whether DNA methylation plays an active role in gene regulation or is a consequence of transcription remains debatable (Schtibeler, 2012; Jones et al., 2013). Several genome wide studies in both animals and plants showed alteration of promoter methylation status by transcription factor binding (Stadler et al., 2011; Thurman et al., 2012; Zhong et al., 2013). The binding sites of RIN, a master MADS-box transcription factor for ripening in tomato, becomes hypomethylated during the ripening process when RIN starts to be expressed (Zhong et al., 2013). In the case of FT, when the CArG boxes were targeted by IRs (FTᵢₙ₁-Ri), cytosines within the CArG boxes were heavily methylated (FIG. 2f) and FLC binding to this region was reduced (FIG. 3b). These data indicate that DNA methylation plays an active role in regulating transcription factor binding to its target site. When the IRs targeted DNA sequences outside the CArG boxes, the FLC accumulation in transgenic plants (FTᵢₙ₁ₛ-Ri-2) remained unchanged compared to WT (FIG. 3h). Methylation levels within the binding sites were significantly lower than nearby cytosines regardless of a clear methylation spreading beyond this region (FIG. 3g). It does not appear that the hypomethylation was caused by reduced targeting smRNAs based on the following considerations: (1) the nearest upstream and downstream cytosines which were just 23 and 15 by away were heavily methylated and (2) these cytosines were as heavily methylated as the nearby cytosines when targeted by IRs, suggesting this region could produce smRNAs equally. MADS-box transcription factors, such as SVP, MAF3, etc., can form dimers or higher-order multimers with or without FLC to bind to the FT CArG boxes (Helliwell et al., 2006; Lee et al., 2007; Gu et al., 2013). This factor may account for the slightly elevation of methylation of the CArG boxes by the flc-3 mutation in FTᵢₙ₁ₛ-Ri-2flc3 plants (FIG. 11). It can be concluded that cytosines bound by transcription factors could be protected from the DNA methylation machinery. The data presented herein suggests that the relationship between DNA methylation and transcription may be determined by the strength of input signals.

DNA methylation could regulate gene expression by altering chromatin structure (Razin, 1998). In animals, DNA methyltransferase DNMT3a directly interacts with H3K9 histone methylatransferase SUV39H1 and DNA methylation and H3K9me3 are co-localized in vivo (Fuks et al., 2003). In Arabidopsis, DNA methylation and H3K9 methylation are linked by a sub family of SET domain H3K9 methyltransferases (SUVHs) which bind to methylated DNA through the SRA domain (Johnson et al., 2007). As shown above, the H3K9me3 levels were significantly higher on the targeted region in activated plants than in control plants (FIG. 4c). A genome wide epigenomic study has shown that H3K9me3 is mainly distributed in gene body regions consistent with this histone marked playing a role in tranascripiton elongation in Arabidopsis (Roudier et al., 2011). H3K9me3 dynamics is positively correlated with GL2 expression in cell fate specification (Caro et al., 2004), and genome wide transcriptional activities during deetiolation Charron et al., 2009) suggests that gene body H3K9me3 may promote transcription in normal plant developmental processes. Gene body H3K9me3 may perform a conserved function in regulating transcription elongation since this marker has been reported to be associated with transcription elongation in mammals (Vakoc et al., 2005). As previously reported with the TMM (too many mouths) gene (ref 12), FT was silenced when the DNA methylation and H3K9me3 were introduced by IRs targeting on the promoter regions (FIGS. 13a-13c). The data herein clearly demonstrate that the same type of epigenetic modification can lead to opposite genetic outcome depending on the location of the modifications on the gene locus.

Overexpression is an important strategy to study gene function, especially when a knock-out mutant is not available. However, the significance of overexpression phenotype is largely dependent on the promoter used to transcribe the target gene. The widely used cauliflower mosaic virus 35S promoter as well as other constitutive promoter used in plant studies elevated transgene expression but compromised the tempo-spatial expression patterns of the native gene (Benfy and Chua, 1990). Overexpression of a gene product in inappropriate cell types may lead to artifacts or toxic lethality. In the work described here, we activated gene expression without introduce an extra-copy of the target gene; most important, the tempo-spatial expression of targeted gene was maintained. This strategy may facilitate us to decipher gene function by tissue-specific biotechnology applications overexpression.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

It will be appreciated that the methods and compositions of the instant invention can be incorporated in the form of a variety of embodiments, only a few of which are disclosed herein. Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### BIBLIOGRAPHY

Adrian, J. et al. (2010). cis-Regulatory elements and chromatin state coordinately control temporal and spatial expression of FLOWERING LOCUS T in Arabidopsis. The Plant Cell 22, 1425-1440.

Agorio, A., and Vera, P. (2007). ARGONAUTE4 is required for resistance to Pseudomonas syringae in Arabidopsis. The Plant Cell 19, 3778-3790.

Ball, M. P. et al. (2009). Targeted and genome-scale strategies reveal gene-body methylation signatures in human cells. Nature biotechnology 27, 361-368.

Baulcombe, D. C. (1996). RNA as a target and an initiator of post-transcriptional gene silencing in transgenic plants. Plant molecular biology 32, 79-88.

Benfey, P. N. and Chua, N.-H. (1990). The cauliflower mosaic virus 35S promoter: combinatorial regulation of transcription in plants. Science 250, 959-966.

Bonfim, K. et al. (2007). RNAi-mediated resistance to Bean golden mosaic virus in genetically engineered common bean (Phaseolus vulgaris). Mol Plant Microbe Interact 20, 717-726.

Caro, E. et al. (2007) A chromatin link that couples cell division to root epidermis patterning in Arabidopsis. Nature 447, 213-217.

Charron, J.-B. F. et al. (2009). Dynamic landscapes of four histone modifications during deetiolation in Arabidopsis. Plant Cell 21, 3732-3748.

Chiu, W. et al. (1996). Engineered GFP as a vital reporter in plants. Current Biology 6, 325-330.

Christensen, A. H. and Quail, P. H. (1989). Sequence analysis and transcriptional regulation by heat shock of polyubiquitin transcripts from maize. Plant Mol Biol 12, 619-632.

Christensen, A. H. et al. (1992). Maize polyubiquitin genes: structure, thermal perturbation of expression and transcript splicing, and promoter activity following transfer to protoplasts by electroporation. Plant Mol Biol 18, 675-689.

Clough, S. J., and Bent, A. F. (1998). Floral dip: a simplified method for Agrobacterium-mediated transformation ofArabidopsis thaliana. The plant journal 16, 735-743

Daxinger, L. et al. (2009). A stepwise pathway for biogenesis of 24-nt secondary siRNAs and spreading of DNA methylation. The EMBO Journal 28, 48-57.

Deng, S. et al. (2014). Transcriptional Silencing of Arabidopsis Endogenes by Single-Stranded RNAs Targeting the Promoter Region. Plant and Cell Physiology 55, 823-33.

De Wet, J. R. et al. (1987). Firefly luciferase gene: structure and expression in mammalian cells. Mol Cell Biol 7, 725-737.

Fuentes, A. et al. (2006). Intron-hairpin RNA derived from replication associated protein Cl gene confers immunity to tomato yellow leaf curl virus infection in transgenic tomato plants. Transgenic Res 15, 291-304.

Fuks, F. et al. (2003). The DNA methyltransferases associate with HP1 and the SUV39H1 histone methyltransferase. Nucleic acids research 31, 2305-2312.

Goff, S. A. et al. (1990). Transactivation of anthocyanin biosynthetic genes following transfer of B regulatory genes into maize tissues. EMBO J 9, 2517-2522.

Gu, X. et al. (2013). Arabidopsis FLC clade members form flowering-repressor complexes coordinating responses to endogenous and environmental cues. Nature communications 4.

Guo, H. S. et al. (2003). A chemical-regulated inducible RNAi system in plants. Plant J 34, 383-392.

Hamilton, A. et al. (2002). Two classes of short interfering RNA in RNA silencing. The EMBO Journal 21, 4671-4679.

Helliwell, C. A. et al. (2006). The Arabidopsis FLC protein interacts directly in vivo with SOCl and FT chromatin and is part of a high-molecular-weight protein complex. The Plant Journal 46, 183-192.

Hetzl, J. et al. (2007). CyMATE: a new tool for methylation analysis of plant genomic DNA after bisulphite sequencing. The Plant journal : for cell and molecular biology 51, 526-536.

Hu, S. et al. (2013). DNA methylation presents distinct binding sites for human transcription factors. Elife 2, e00726.

Jefferson, R. A. et al. (1991). Plant Molecular Biology Manual, ed. Gelvin et al., Kluwer Academic Publishers, pp. 1-33.

Johnson, L. M. et al. (2007). The SRA methyl-cytosine-binding domain links DNA and histone methylation. Current Biology 17, 379-384.

Jones, L. et al. (1999). RNA-DNA interactions and DNA methylation in post-transcriptional gene silencing. The Plant Cell 11, 2291-2301.

Jones, M. J. et al. (2013). DNA methylation, genotype and gene expression: who is driving and who is along for the ride? Genome Biol 14, 126.

Jones, P. A. (2012). Functions of DNA methylation: islands, start sites, gene bodies and beyond. Nature Reviews Genetics 13, 484-492.

Kain, S. R. et al. (1995). Green fluorescent protein as a reporter of gene expression and protein localization. Biotechniques 19, 650-655.

Karimi, M. et al. (2002). GATEWAYTM vectors for Agrobacterium-mediated plant transformation. Trends in plant science 7, 193-195.

Kass, S. U. et al. (1997). How does DNA methylation repress transcription? Trends in Genetics 13, 444-449.

Kinoshita, Y. et al. (2007). Control of FWA gene silencing in Arabidopsis thaliana by SINE-related direct repeats. The Plant Journal 49, 38-45.

Last, D. I. et al. (1991). pEmu: an improved promoter for gene expression in cereal cells. Theor Appl Genet 81, 581-588.

Law, J. A., and Jacobsen, S.E. (2010). Establishing, maintaining and modifying DNA methylation patterns in plants and animals. Nature Reviews Genetics 11, 204-220.

Lee, J. H. et al. (2007). Role of SVP in the control of flowering time by ambient temperature in Arabidopsis. Genes & Development 21, 397-402.

Matzke, M. et al. (2009). RNA-mediated chromatin-based silencing in plants. Current opinion in cell biology 21, 367-376.

McElroy, D. et al. (1990). Isolation of an efficient actin promoter for use in rice transformation. Plant Cell 2, 163-171.

Mette, M. et al. (2000). Transcriptional silencing and promoter methylation triggered by double-stranded RNA. The EMBO Journal 19, 5194-5201.

Michaels, S. D. and Amasino, R. M. (1999). FLOWERING LOCUS C encodes a novel MADS domain protein that acts as a repressor of flowering. The Plant Cell 11, 949-956.

Morel, J.-B. et al. (2000). DNA methylation and chromatin structure affect transcriptional and post-transcriptional transgene silencing in Arabidopsis. Current Biology 10, 1591-1594.

Morel, J.-B. et al. (2002). Fertile hypomorphic ARGONAUTE (agol) mutants impaired in post-transcriptional gene silencing and virus resistance. The Plant Cell 14, 629-639.

Odell, J. T. et al. (1985). Identification of DNA sequences required for activity of the cauliflower mosaic virus 35S promoter. Nature 313, 810-812.

Razin, A. (1998). CpG methylation, chromatin structure and gene silencing—three-way connection. The EMBO Journal 17, 4905-4908.

Roudier, F. et al. (2011). Integrative epigenomic mapping defines four main chromatin states in Arabidopsis. EMBO journal 30, 1928-1938.

Schiibeler, D. (2012). Epigenetic Islands in a Genetic Ocean. Science 338, 756-757.

Shibuya, K. et al. (2009). RNA-directed DNA methylation induces transcriptional activation in plants. Proceedings of the National Academy of Sciences 106, 1660-1665.

Smith, N. A. et al. (2000). Total silencing by intron-spliced hairpin RNAs. Nature 407:319-320.

Stadler, M. B. et al. (2011). DNA-binding factors shape the mouse methylome at distal regulatory regions. Nature 480, 490-495.

Thurman, R. E. et al. (2012). The accessible chromatin landscape of the human genome. Nature 489, 75-82.

Turck, F. et al. (2008). Regulation and identity of florigen: FLOWERING LOCUS T moves center stage. Annu. Rev. Plant Biol. 59, 573-594.

Vakoc, C. R. et al. (2005). Histone H3 lysine 9 methylation and HPlgamma are associated with transcription elongation through mammalian chromatin. Molecular cell 19, 381-391

Vanderschuren, H. et al. (2007a). Transgenic cassava resistance to African cassava mosaic virus is enhanced by viral DNA-A bidirectional promoter-derived siRNAs. Plant Mol Biol 64, 549-557.

Vanderschuren, H. et al. (2007b). Engineering resistance to geminiviruses--review and perspectives. Plant Biotechnology Journal 5, 207-220.

Velten, J. et al. (1984). Isolation of a dual plant promoter fragment from the Ti plasmid of Agrobacterium tumefaciens. EMBO J 3, 2723-2730.

Wang, M. B. et al. (2000). A single copy of a virus-derived transgene encoding hairpin RNA gives immunity to barley yellow dwarf virus. Mol Plant Pathol 1, 347-356.

Wassenegger, M. et al. (1994). RNA-directed de novo methylation of genomic sequences in plants. Cell 76, 567-576.

Yu, D.-H. et al. (2013). Developmentally programmed 3′ CpG island methylation confers tissue-and cell-type-specific transcriptional activation. Molecular and cellular biology 33, 1845-1858.

Zhang, X. et al. (2006). Genome-wide high-resolution mapping and functional analysis of DNA methylation in arabidopsis. Cell 126, 1189-1201.

Zhong, S. et al. (2013). Single-base resolution methylomes of tomato fruit development reveal epigenome modifications associated with ripening. Nature biotechnology 31, 154-159.

Zilberman, D. et al. (2007). Genome-wide analysis of Arabidopsis thaliana DNA methylation uncovers an interdependence between methylation and transcription. Nature genetics 39, 61-69.

Zrachya, A. et al. (2007). Production of siRNA targeted against TYLCV coat protein transcripts leads to silencing of its expression and resistance to the virus. Transgenic Res 16, 385-398.

Zuo, J. et al. (2001). Chemical-regulated, site-specific DNA excision in transgenic plants. Nat Biotechnol 19, 157-161.

What is claimed is:

1. A nucleic acid construct comprising a plant operable promoter operatively linked to a nucleic acid fragment that is substantially homologous to a target region of an intron of a target gene in a plant.

2. The nucleic acid construct of claim 1, further comprising a plant operable promoter operatively linked to a second nucleic acid fragment that is substantially homologous to a target region of an intron of a second target gene in a plant.

3. The nucleic acid construct of claim 1, wherein the nucleic acid fragment is operatively linked in a sense orientation to the promoter and is further linked to the same fragment in an antisense orientation.

4. The nucleic acid construct of claim 1, wherein the nucleic acid fragment is operatively linked in an antisense orientation to the promoter and is further linked to the same fragment in a sense orientation.

5. The nucleic acid construct of claim 3, wherein the nucleic acid fragment in the sense orientation is linked by a spacer nucleotide sequence to the nucleic acid fragment in the antisense orientation.

6. The nucleic acid construct of claim 3, wherein the nucleic acid fragment in the sense orientation is linked by a spacer nucleotide sequence to the nucleic acid fragment in the antisense orientation.

7. The nucleic acid construct of claim 3, wherein the nucleic acid fragment in the sense orientation is linked by an intron to the nucleic acid fragment in the antisense orientation.

8. The nucleic acid construct of claim 4, wherein the nucleic acid fragment in the antisense orientation is linked by an intron to the nucleic acid fragment in the sense orientation.

9. A vector comprising the nucleic acid construct of claim 1.

10. A transgenic plant cell comprising the nucleic acid construct of claim 1.

11. A transgenic plant seed comprising the nucleic acid construct of claim 1.

12. A transgenic plant comprising the nucleic acid construct of claim 1.

13. A transgenic plant having increased expression of a target gene, wherein the increased expression is achieved by introducing into a cell of the plant the nucleic acid construct of claim 1 that when expressed in the plant cell produces a dsRNA molecule that activates expression of the target gene.

14. The transgenic plant of claim 14, wherein the dsRNA is a hpRNA or an ihpRNA.

15. A method of preparing a transgenic plant having increased expression of a target gene, the method comprises stably incorporating the nucleic acid construct of claim 1 in the genome of a plant, wherein the nucleic acid construct encodes a dsRNA molecule that when expressed in a transgenic plant activates expression of the target gene.

16. The method of claim 15, wherein the dsRNA is a hpRNA or an ihpRNA.

17. A method of conferring increased target gene expression in a transgenic plant, the method comprising expressing the nucleic acid construct of claim 1 in a transgenic plant, wherein the nucleic acid construct encodes a dsRNA molecule that when expressed in the transgenic plant activates expression of the target gene.

18. The method of claim 17, wherein the dsRNA is a hpRNA or an ihpRNA.

19. A method of altering the phenotype of a transgenic plant, the method comprises stably incorporating the nucleic acid construct of claim 1 in the genome of a plant, wherein the nucleic acid construct encodes a dsRNA molecule that when expressed in a transgenic plant activates or increases expression of a target gene which confers the altered phenotype of the transgenic plant.

20. A method of preparing a non-transgenic plant exhibiting an increased expression or activation of an endogenous target gene and/or an altered phenotype, the method comprises
(a) stably incorporating the nucleic acid construct of claim 1 in the genome of a plant, wherein the nucleic acid construct encodes a dsRNA molecule that when expressed in a transgenic plant activates expression of the endogenous target gene which confers the altered phenotype of the transgenic plant,
(b) crossing the transgenic plant of step (a) with a non-transgenic plant to produce progeny, and
(c) selecting progeny in which the incorporated nucleic acid construct of claim 1 has been eliminated due to segregation and which exhibits an increased activation of an endogenous target gene and/or an altered phenotype,
or the method comprises
(a) transiently incorporating the nucleic acid construct of claim 1 in plant cells, wherein the nucleic acid construct encodes a dsRNA molecule,
(b) selecting a transgenic plant cell of step a) in which the expression of the endogenous target gene is activated,
(c) proliferating the plant cell of step (b) to produce a population of plant cells in which the expression of the endogenous target gene is activated,
(d) selecting a plant cell from the population of step (c) in which the expression of the endogenous target gene is activated and which does not contain the nucleic acid construct of claim 1, and
(e) regenerating a non-transgenic plant from the selected cell of step (d), wherein the plant exhibits an increased activation of an endogenous target gene and/or an altered phenotype,
or the method comprises
(a) introducing siRNA or smRNA produced by a nucleic acid construct of claim 1 into a plant cell,
(b) selecting a plant cell of step (a) in which the expression of the endogenous target gene is activated, and
(c) regenerating a non-transgenic plant from the selected cell of step (b), wherein the plant exhibits an increased activation of an endogenous target gene and/or an altered phenotype.

21. A plant prepared by the method of claim 20.
